(19) United States
(12) Patent Application Publication
(10) Pub. No.: US 2024/0115600 A1
(43) Pub. Date: Apr. 11, 2024

(54) ORAL GRANULES WITH COMBINATION OF EXTENDED-RELEASE AND QUICK-RELEASE MAGNESIUM, AND PRODUCTION METHODS

(71) Applicant: Hermes Pharma GmbH, Pullach (DE)

(72) Inventors: Gerd WEISS, München (DE); Josef HAALA, Pullach (DE); Wolfgang SCHIEMENZ, Pullach (DE)

(21) Appl. No.: 18/262,183

(22) PCT Filed: Oct. 4, 2022
(86) PCT No.: PCT/EP2022/077586

§ 371 Date: Jul. 19, 2023

(30) Foreign Application Priority Data
Oct. 4, 2021 (EP) 21200711.6

(51) Int. Cl. A61K 33/06; A61K 9/28

(52) U.S. Cl. CPC A61K 33/06; A61K 9/282

(57) ABSTRACT

The invention enables direct oral granules enabling controlled release of magnesium, having both extended-release and non-extended-release fractions. The granules include extended-release particles having a magnesium-containing core and a lipophilic coating, with at least the cores of these extended-release particles having a mandated abrasion resistance which is determined by means of vibration screening over a period of 60 minutes and which allows these cores to be melt-coated, more particularly melt-coated with lipids in a fluidized bed. The invention further provides a method for characterizing the magnesium-containing cores, and also a process for producing the extended-release particles by means of hotmelt coating.

### BACKGROUND OF THE INVENTION

The present invention relates to new compositions, especially direct oral granules, which comprise the active ingredient magnesium, more particularly to a combination of a magnesium in extended-release form and also in a quicker-releasing, non-extended-release form. The uses of the compositions include nutritional supplements and/or drugs in the therapy or prevention of magnesium deficiency states.

In order to ensure sufficient supply of magnesium to the body even in phases of increased demand (in cases, for example, of stress, pregnancy, nutritional deficiency, high-level sporting activity, diuretic therapy, or severe alcohol consumption), it is sometimes advisable to substitute this important mineral with external sources. As with many water-soluble active ingredients, there is a risk here of the body rapidly excreting excess magnesium in the urine, thus excreting those amounts which it is unable to use in the very short term or to house in endogenous stores. Moreover, certain persons respond to higher concentrations of free magnesium in the gastrointestinal tract with stomach complaints and/or diarrhea. A formulation having at least partly controlled release of the total magnesium dose it contains can provide remedy here and also offers further advantages such as a reduced frequency of administration, among others.

For a lasting and very largely constant supply of magnesium to the body, formulations with controlled release have already been developed, as for example with extended release (delayed release) or pulsatile release (i.e., release in a plurality of “pulses” over a prolonged period). On the market, for example, is a three-layer magnesium tablet with added B vitamins (Biolectra® Magnesium 400 mg ultra 3-phase depot; Hermes Arzneimittel GmbH), which is conceived as a nutritional supplement to be taken once daily. The three layers contain (i) 200 mg of magnesium (+Vit. B2 and B12) for immediate release, (ii) 90 mg of magnesium (+Vit. B1) for intermediate release, and (iii) 110 mg of magnesium (+Vit. B6) for long-term release. Magnesium compounds used are magnesium oxide, magnesium carbonate, and magnesium citrate. Excipients used for the extended release are swellable polymers such as hydroxypropylmethylcellulose (HPMC), hydroxypropylcellulose (HPC) and polyvinylpyrrolidone (PVP). Unfortunately, these tablets are relatively large, owing to the high required quantities of the magnesium compounds and also of the excipients for their extended release (around 2 g tablet weight), and for many users they therefore cannot be swallowed comfortably or without problems.

One possible remedy, or alternative, for large, difficult-to-swallow tablets offered by effervescent tablets or powders for dissolving in water, or else what are called direct oral granules. Granules in the narrowest sense are accumulations which are produced via aggregation or agglomeration of fine and/or coarse powders and are therefore usually more coarse-grained than fine and coarse powders (the boundaries of the particle sizes, especially of coarse powders and granules, can overlap). Common processes for granulation/aggregation are known to the skilled person and described in the literature. The term “direct oral granules”, on the other hand, is understood more broadly by the skilled person; in this case it is usually immaterial whether the accumulation has actually been obtained by aggregation of powder particles, or whether individual or multiple constituents of the oral granules have been otherwise processed. Direct oral granules are primarily flowable accumulations which can be administered orally, preferably having a particle size in the range from 50 μm to 500 μm, which are employed or administered directly, namely in their granular or particulate form; consequently, they do not require water or other drinkable liquids to wash them down for their administration. They can be scattered, for example, in the mouth and/or on the tongue, and then easily swallowed.

For such direct oral granules, however, the mouthfeel is very important, and is determined by factors including structural properties (e.g., textures such as fatty, dry, hard, formable, sticky, crumbly, etc.), by geometric properties (large/small, round/angular, etc.), and also by properties associated with the perceived water content in the mouth and throat (juicy, dry, crisp, or the like), with temperature sensations (warming, cooling) or with the stimulation of free nerve ends (sharp, stinging, tingling). Lipid-based hotmelt coatings have become established here, as they frequently leave behind less of a “foreign-body” sensation on the tongue.

For example, EP3042648B1 describes direct oral granules with extended-release particles of ascorbic acid, composed of crystalline ascorbic acid and a lipid-based coating which is applied as a melt in a fluidized bed to the crystalline ascorbic acid.

The development of industrially producible direct oral granules which enable controlled release of magnesium, with both extended-release and non-extended-release fractions, has hitherto, however, been made more difficult for reasons including the fact that some of the commercially available unfinished product for magnesium formulations—in contrast, for example, to crystalline ascorbic acid—lack sufficient physical stability to withstand the hotmelt coating process. In such processes, a very frequent observation has been, for example, that pregranulated product, under the mechanical loading of the fluidized bed and/or under the heat effect inherent to the process, fell apart and then clogged the filters of the fluidized bed apparatus, well before the hotmelt coating process had advanced to a sufficient point. The need for very frequent filter changes, and/or filter cleaning steps, made these processes unsuitable, therefore, for the industrial scale (e.g., ≥50 kg per batch).

Furthermore, it is exactly those magnesium compounds which are more appropriate for the non-extended-release dose fraction, by virtue of their higher solubility and bioavailability (e.g., magnesium citrates or similar magnesium salts of organic acids), which, in a majority of cases, are very acidic or less acceptable from a taste standpoint, and are therefore more difficult to administer as direct oral granules, as these granules come into direct contact, undiluted, with the tongue.

The present invention is therefore based on the object of providing a composition for magnesium compounds that can be economically produced industrially with few steps and which permits controlled release of magnesium, having both extended-release and non-extended-release fractions. The composition ought to be easier and more convenient to administer than customary tablets or capsules, preferably without additional administration of liquid as well. It ought, furthermore, to ensure the stability of the magnesium compounds. A further object of the invention, for magnesium-containing cores that are intended for lipid coating, and more particularly for a process of hotmelt coating with lipids in a fluidized bed—such as, for example, commercially available unfinished magnesium-containing core products—, is to characterize these cores beforehand such that their serviceability, or suitability, for such a coating process can be determined and excessive losses of material due to unstable core material can be avoided.

This object is achieved by the solid pharmaceutical or nutraceutical composition of the invention in the form of direct oral granules as described below, and also by the methods and processes for their production, as likewise described below.

### SUMMARY OF THE INVENTION

In a first aspect, the present invention relates to a solid pharmaceutical or nutraceutical composition in the form of direct oral granules with at least dual release of active ingredient, comprising components as follows:
(a) coated, extended-release particles having a magnesium-containing core and a lipid coating, the core comprising or consisting of a magnesium compound, and the extended-release particles releasing the comprised magnesium in an extended manner;
(b) non-extended-release, magnesium-containing particles, the particles comprising or consisting of a magnesium compound, and the particles releasing the comprised magnesium in a non-extended manner;
(c) one or more water-soluble excipients selected from the group of sugars, sugar alcohols, and oligosaccharides; and
(d) optionally one or more further excipients;
wherein at least the magnesium-containing cores of the extended-release particles of component (a), and/or a sample thereof, have an abrasion resistance such that after vibration screening over a period of 60 minutes:
   the fine fraction of particles having a screen diameter of <200 μm after a screening time of 60 minutes, t₆₀ₘₘ, is higher by not more than 100 wt. % relative to the initial fine fraction of this screen diameter after a screening time of 5 minutes, t₅ₘᵢₙ; and/or
   the fraction of particles having a screen diameter of 200 μm≤x<800 μm after a screening time of 60 minutes, t₆₀ₘᵢₙ, is lower by not more than 18 wt. % relative to the initial fraction of this screen diameter after a screening time of 5 minutes, t₅ₘᵢₙ.

In a second aspect, the present invention relates to a method for characterizing a magnesium-containing core for producing a solid, lipid-coated pharmaceutical or nutraceutical composition in a fluidized bed (optionally the production of the extended-release particles of component (a) of the composition according to the first aspect of the invention), the core comprising or consisting of a magnesium compound, and wherein the method comprises being carried out by means of vibration screening, and by magnesium-containing cores which are characterized as being suitable for producing the solid, lipid-coated pharmaceutical or nutraceutical composition in a fluidized bed in this test having an abrasion resistance such that after the vibration screening over a period of 60 minutes:
the fine fraction of particles having a screen diameter of <200 μm after a screening time of 60 minutes, t₆₀ₘᵢₙ, is higher by not more than 100 wt. %, preferably by not more than 50 wt. %, more preferably by not more than 20 wt. %, relative to the initial fine fraction of this screen diameter after a screening time of 5 minutes, t₅ₘᵢₙ; and/or
the fraction of particles having a screen diameter of 200 μm≤x<800 μm after a screening time of 60 minutes, t₆₀ₘᵢₙ, is lower by not more than 18 wt. %, preferably by not more than 15 wt. %, more preferably by not more than 12 wt. %, relative to the initial fraction of this screen diameter after a screening time of 5 minutes, t₅ₘᵢₙ.

In a third aspect, the present invention relates to a process for producing a solid pharmaceutical or nutraceutical composition according to the first aspect of the invention, wherein the process contains or comprises the following steps:
(i) providing a magnesium-containing core, the core comprising or consisting of a magnesium compound;
(ii) providing a molten coating material comprising a lipid;
(iii) fluidizing the magnesium-containing cores;
(iv) spraying the fluidized magnesium-containing cores with the molten coating material;
(v) cooling the coated magnesium-containing cores, so that the lipid solidifies and extended-release particles of component (a) are obtained which release the comprised magnesium in an extended manner; and
(vi) mixing the extended-release particles obtained in this way as component (a) with the following further components:
   (b) non-extended-release particles having a second magnesium-containing core, the particle comprising or consisting of a magnesium compound, and the particles releasing the comprised magnesium in a non-extended manner;
   (c) one or more water-soluble excipients selected from the group of sugars, sugar alcohols, and oligosaccharides; and
   (d) optionally further excipients;
   wherein at least the magnesium-containing cores of the extended-release particles of component (a), and/or a sample thereof, have an abrasion resistance such that after vibration screening over a period of 60 minutes:
      the fine fraction of particles having a screen diameter of <200 μm after a screening time of 60 minutes, t₆₀ₘᵢₙ, is higher by not more than 100 wt. % relative to the initial fine fraction of this screen diameter after a screening time of 5 minutes, t₅ₘᵢₙ; and/or
      the fraction of particles having a screen diameter of 200 μm≤x<800 μm after a screening time of 60 minutes, t₆₀ₘᵢₙ, is lower by not more than 18 wt. % relative to the initial fraction of this screen diameter after a screening time of 5 minutes, t₅ₘᵢₙ.

In other words, in the production process according to the third aspect of the invention, the magnesium-containing cores provided in step (i) are of the kind which have been characterized, by means of the characterization method according to the second aspect of the invention, as being suitable for producing a solid, lipid-coated pharmaceutical or nutraceutical composition in a fluidized bed. This characterization is typically accomplished on a sample basis, such as for a defined batch of the magnesium-containing cores, for example.

In a further aspect of the invention, the present invention relates to a solid pharmaceutical or nutraceutical composition in the form of direct oral granules with at least dual release of active ingredient, produced by means of the process according to the third aspect of the invention.

### BRIEF DESCRIPTION OF THE FIGURES

FIG. 1a and FIG. 1b show the results of vibration screening over a time of 60 minutes for two of the unfinished granulated magnesium oxide (MgO) core products tested (here ‘Heavy Magnesium Oxide EP’ from Kyowa Chemical Industry Co., Ltd Japan, identified hereinafter as ‘MgO 1’; and MagGran® MO from Magnesia GmbH Deutschland; hereinafter ‘MgO 2’).

FIG. 2 shows the effect of the formulation on the release of magnesium in 0.1 N hydrochloric acid at 37° C. for uncoated MgO cores, coated MgO cores (coating level 15 wt. %) and direct oral granules (ODG) of the invention that include these coated MgO cores.

FIG. 3 shows the effect of the coating level on the release of magnesium in 0.1 N hydrochloric acid at 37° C. for uncoated MgO cores, and for direct oral granules (ODG), of the invention that include coated MgO cores with a coating level of 10 wt. % (ODG 3) and 15 wt. % (ODG 1 and ODG 2).

FIG. 4 shows the effect of the formulation on the release of magnesium in 0.1 N hydrochloric acid at 37° C. for uncoated MgO cores, and for direct oral granules (ODG), of the invention that include coated MgO cores with a coating level of 10 wt. % and also different fractions of non-extended-release magnesium, with ODG 3 containing less non-extended-release magnesium than ODG 4.

### DETAILED DESCRIPTION OF THE INVENTION

In a first aspect, the present invention relates to a solid pharmaceutical or nutraceutical composition in the form of direct oral granules with at least dual release of active ingredient, comprising components as follows:
(a) coated, extended-release particles having a magnesium-containing core and a lipid coating, the core comprising or consisting of a magnesium compound, and the extended-release particles releasing the comprised magnesium in an extended manner;
(b) non-extended-release, magnesium-containing particles, the particles comprising or consisting of a magnesium compound, and the particles releasing the comprised magnesium in a non-extended manner;
(c) one or more water-soluble excipients selected from the group of sugars, sugar alcohols, and oligosaccharides; and
(d) optionally one or more further excipients;
wherein at least the magnesium-containing cores of the extended-release particles of component (a), and/or a sample thereof, have an abrasion resistance such that after vibration screening over a period of 60 minutes:
   the fine fraction of particles having a screen diameter of <200 μm after a screening time of 60 minutes, t₆₀ₘᵢₙ, is higher by not more than 100 wt. % relative to the initial fine fraction of this screen diameter after a screening time of 5 minutes, t₅ₘᵢₙ; and/or
   the fraction of particles having a screen diameter of 200 μm≤x<800 μm after a screening time of 60 minutes, t₆₀ₘᵢₙ, is lower by not more than 18 wt. % relative to the initial fraction of this screen diameter after a screening time of 5 minutes, t₅ₘᵢₙ.

In connection with the invention, the term ‘extended-release particles’ designates particles which comprise an active ingredient, here a magnesium compound, and at least one excipient in the form of a coating, and whose construction is such that the coating of excipient results in slow release of the active ingredient (so-called reservoir systems). In the case of the present invention, this extended release is ensured by a lipid coating.

In one of the preferred embodiments, the lipid coating of the extended-release particles of component (a) is applied to the magnesium-containing cores by a hotmelt coating process, or in other words, it is a lipid hotmelt coating.

Since the present invention relates to solid pharmaceutical or nutraceutical compositions in the form of direct oral granules, the term ‘core’ or ‘magnesium-containing core’ as used herein refers to core materials in accumulation form, examples being unfinished granular products in dry, flowable particle form, having average particle sizes of typically around 600 μm or less.

Furthermore, in connection with the invention described herein, the term ‘abrasion resistance’ designates not only those fine fractions of an accumulation that have come about exclusively by surface abrasion but also those which have come about by the falling-apart or fracture of the parent tested particles or cores. The principal reason for this is that these two fractions (abraded material in the narrower sense, and fractured material) are indistinguishable following the screen analysis. The concepts of abrasion resistance and fracture resistance are therefore to be understood as being synonymous for this invention.

In one specific embodiment, at least the magnesium-containing cores of the extended-release particles of component (a), and/or a sample thereof, have an abrasion resistance such that after vibration screening over a period of 60 minutes, the fine fraction of particles having a screen diameter of <200 μm after a screening time of 60 minutes, t₆₀ₘᵢₙ, is higher by not more than 50 wt. %, preferably by not more than 20 wt. %, relative to the initial fine fraction of this screen diameter after a screening time of 5 minutes, t₅ₘᵢₙ; and/or the fraction of particles having a screen diameter of 200 μm≤x<800 μm after a screening time of 60 minutes, t₆₀ₘᵢₙ, is lower by not more than 15 wt. %, preferably by not more than 12 wt. %, relative to the initial fraction of this screen diameter after a screening time of 5 minutes, t₅ₘᵢₙ.

In one further embodiment, at least the magnesium-containing cores of the extended-release particles of component (a), and/or a sample thereof, have an abrasion resistance such that after vibration screening over a period of 60 minutes, the fraction of particles having a screen diameter of <300 μm after a screening time of 60 minutes, t₆₀ₘᵢₙ, is higher by not more than 55 wt. %, preferably by not more than 35 wt. %, more preferably by not more than 15 wt. %, relative to the initial fraction of this screen diameter after a screening time of 5 minutes, t₅ₘᵢₙ; and/or the fraction of particles having a screen diameter of 300 μm≤x<800 μm after a screening time of 60 minutes, t₆₀ₘᵢₙ, is lower by not more than 30 wt. %, preferably by not more than 27 wt. %, more preferably by not more than 24 wt. % relative to the initial fraction of this screen diameter after a screening time of 5 minutes, t₅ₘᵢₙ.

It is of course also possible, optionally, for the non-extended-release magnesium-containing particles, and/or a sample thereof, to have an abrasion resistance as described above. This would be an advantage, for instance, if the application of a water-soluble, non-extended-release coating to the non-extended-release magnesium-containing particles, or granulation thereof in the fluidized bed, for example, was considered.

More precise parameters of the vibration screening with which the above-described abrasion resistance is determined, and/or the magnesium-containing cores are characterized, are described later on below for the second aspect of the invention, in more detailed form; all of these parameters are explicitly applicable to the composition according to the first aspect of the invention.

In an illustratively summarizing embodiment, for example, the vibration screening may be carried out with 100 g of the magnesium-containing cores (total amount for screening that is applied to the topmost screen in the tower, for example, an 800 μm screen) and with 1 g of screening aid per screen tray, and also at a vibration amplitude of 80/min. The screening tower in this case that is employed may be a standard commercial screening tower having screen trays of around 20 cm in diameter, an example being the AS 200 basic from Retsch GmbH. The screening aid used here may comprise, for example, abrasion-resistant silica gel beads which have a diameter of around 2-6 mm, or around 2-5 mm, or around 2-4 mm (e.g., around 3 mm), and a bulk density of around 650-850 kg/m³, or around 700-800 kg/m³ (e.g., a bulk density of approximately 750 kg/m³). The duration of the vibration screening of the invention has been set at 60 min (with at least one intermediate weighing at 5 min), since this corresponds approximately to the timeframe of a common hotmelt coating process (a variant of the application of lipid coatings); in other words, as long as the cores are subject in general to the mechanical stress of the fluidized bed, and also to the typically elevated temperatures of a hotmelt coating process, and must have a sufficient abrasion resistance. In cases requiring a significantly longer spraying process, it would be possible theoretically to conceive of a longer screening duration than 60 minutes. Frequently, however, this is not necessary, since in general the magnesium-containing cores after 60 minutes are already coated to such an extent that the coating stabilizes the cores and in particular the effects of the mechanical stress on the core material become smaller and smaller.

As mentioned above, the development of industrially producible direct oral granules with hotmelt-coated magnesium cores had in the past been made more difficult or impossible by the fact that many of the commercially available magnesium formulations did not appear to be sufficiently stable to withstand the hotmelt coating process. For example, a pregranulated product fell apart and/or exhibited severe abrasion, and so clogged the filters of the fluidized bed apparatus well before the hotmelt coating process had advanced to a sufficient point, so necessitating very frequent filter changes and/or filter cleaning steps, and meaning that the process was unsuitable for the industrial scale (e.g., 50 kg per batch). Moreover, the resulting deposits of encrusted abraded core material and sprayed material (e.g., triglycerides such as glycerol tripalmitate and/or glycerol tristearate) on the inner walls of the fluidized bed apparatus were so pronounced and solid that very time-consuming cleaning steps were always necessary.

As part of extensive experiments for the production of the direct oral granules according to the first aspect of the invention, the inventors had initially found commercially available magnesium-containing cores which did not have these problems, or had them to a significantly reduced extent, and which therefore were suitable for a hotmelt coating process with lipids (e.g., with triglycerides such as glycerol tripalmitate and/or glycerol tristearate). Even here, however, there were differences, not only between the unfinished products from different manufacturers but also between different batches from the same manufacturer, and so again it remained unclear whether a particular unfinished magnesium-containing core product was or was not suitable for a hotmelt coating process, especially for a hotmelt coating process on the industrial scale.

Standardized abrasion testers such as a friability tester, for example, were unable, or unable with sufficient accuracy, to classify the magnesium-containing cores of the present invention into cores which were able to withstand a lipid coating process, especially a hotmelt coating process with lipids, in a fluidized bed (including on the industrial scale), and those which did not withstand it and which proved unsuitable for the coating process due to their high level of abrasion.

It was only the use of vibration screening, and particularly the use of vibration screening with the screening aid described, that made it possible to simulate the mechanical loading of a fluidized bed coating process, more particularly that of a fluidized bed coating process in which a hotmelt coating is applied. In this way it is possible beforehand to determine the abrasion resistance of the magnesium-containing cores intended for producing the extended-release particles and so to say whether these cores have sufficient stability for the coating process. As a result, it is possible to avoid unnecessary losses of material and also to reduce the downtimes (in which, for example, the coating equipment must be cleaned), since the batches of magnesium-containing cores that are coated are only those which exhibit the above-described abrasion resistance requirements, or, in other words, the only magnesium-containing cores (or batches thereof) that are coated are those which appear suitable in the method according to the second aspect of the invention. As far as the invention is concerned it is immaterial here whether the abrasion resistance of the magnesium-containing cores intended for producing the extended-release particles (sufficient or not sufficient) is the result of batch variabilities from an individual supplier or is caused by batches from different suppliers.

In one embodiment, the magnesium-containing cores of the extended-release particles of the invention are produced by means of dry compacting, sometimes also referred to as roll compacting.

In one of the preferred embodiment, the magnesium-containing cores of the extended-release particles have a median particle size (D50), measured by means of dynamic image analysis, of between 150 μm and 300 μm, preferably between 165 μm and 285 μm, more preferably between 180 μm and 270 μm.

All of the particle size data used herein—not only measured values but also values derived/calculated from measured values—relate to values ascertained by means of dynamic image analysis, using, for example, the Camsizer® XT apparatus from Retsch Technology GmbH, Haan, Germany, with the X-Jet plug-in cartridge and also the associated analytical software. The Camsizer® setup uses a dynamic imaging technique in which particle samples are dispersed by compressed air, passed through a gap illuminated by pulsed LED light sources, with their images (more precisely, their projections) being recorded at this point by two digital cameras and then analyzed for size and shape. In this way, it is possible to determine a multiplicity of length and width descriptors for the particles, including the mean particle size, the median particle size (D50), and also the D10 and D90 values. The Camsizer® XT apparatus is preferred for the particle size determination in accordance with the invention since it allows the precise and reproducible analysis of particle sizes of fine powders down to 1 μm. However, this should not be misunderstood as ruling out the use of laser scattering or other established methods for particle size determination in the required micrometer range; however, in cases in which the particle sizes ascertained by other processes differ from the values ascertained using the Camsizer®, the values ascertained using the Camsizer® are definitive.

The above-stated particle sizes of the magnesium-containing cores are advantageous in that the resultant corresponding extended-release particles, even after application of the coating needed for extension of release, retain a convenient particle size, which on direct oral administration is not perceived as being coarse-grained or otherwise unpleasant. The particle size of direct oral granules ought in general not to exceed 600 μm, preferably 500 μm; in order to avoid a “foreign-body” sensation in the mouth and to keep the chewing reflex low.

Even unfinished magnesium-containing core products which have been produced via dry compacting offer advantages in this respect, since they often contain no—or at most minimal—admixtures of excipients, and the magnesium-containing cores are therefore present in a concentrated, magnesium-rich form, and so likewise offer small particle sizes. Even with these unfinished products, however, the inventors have also made the above-described observation that the abrasion resistance of the dry-granulated, unfinished products was often not sufficient to allow hotmelt coating processes, including those on the industrial scale. In this regard, the inventors can only speculate whether possibly differences in the residual moisture content of the compressed material, different pressing pressures, the nature of the comminution of the pressed flake, or other factors lead to differences in the abrasion resistance of the unfinished product and so influence, or are factors in influencing, their serviceability for hotmelt coating processes.

In a further preferred embodiment, the magnesium-containing cores of the extended-release particles have a unimodal particle size distribution, i.e., a particle size distribution which has only a single peak in the distribution diagram. This generally suggests a predominantly uniform particle size. Additionally preferred are embodiments in which the magnesium-containing cores of the extended-release particles have a particle size distribution whose narrowness is such that the quotient (D90-D10)/D50 is less than 1.60, preferably less than 1.50, and more preferably less than 1.40. Since it is above D90 and below D10 that the outliers in the particle size distribution tend to be found, a quotient (D90-D10)/D50 of close to one suggests that the majority of the particles have a size close to the median particle size (D50).

It is additionally advantageous that at least the magnesium compound in the extended-release particles of component (a) in its anhydrous form has a magnesium content of 15 wt. % or more, preferably 25 wt. % or more, more preferably 50 wt. % or more. This is an advantage insofar as in this way, the amount and/or size of the “extended-release cores” can be kept low, and the extended-release particles produced from them, even after application of coating, still retain a convenient particle size as described above. Optionally, of course, the magnesium compound in the non-extended-release particles of component (b) in their anhydrous form may also have a magnesium content of 15 wt. % or more, preferably 25 wt. % or more, more preferably 50 wt. % or more. In one embodiment, the magnesium compound in the extended-release particles is selected from magnesium oxide (MgO), magnesium carbonate (MgCO₃), magnesium chloride (MgCl₂), magnesium hydrogen phosphate (MgHPO₄) and/or magnesium acetate (Mg(CH₃COO)₂).

The core of the extended-release particles may optionally comprise one or more excipients—for example, in the case of a pregranulated unfinished magnesium-containing product, a small amount of a water-soluble binder, which may have been added to the unfinished product at the manufacturer end. As mentioned above, however, it is advantageous in this context for as little as possible excipients to have been added, in order in this way to ensure small particle sizes for high magnesium content.

In one embodiment, the magnesium compound in the core of the extended-release particles is magnesium oxide (MgO). In one specific embodiment, the core in the extended-release particles consists essentially of magnesium oxide (MgO).

In one of the preferred embodiment, the coating of the extended-release particles comprises a lipid having a melting point of at least 50° C., or consists essentially thereof, preferably a lipid having a melting point of at least 60° C. Where the coating comprises two or more lipids, at least one of the lipids has a melting point of at least 50° C., preferably at least 60° C. Furthermore, in the not unusual event of a lipid having a melting range rather than a sharp melting point, the melting point in the sense of the invention refers to the lower limit of the melting range, in other words the temperature at which the lipid begins to melt during heating.

The inventors have determined that, in particular, lipids having a relatively high melting point of at least 50° C., preferably at least 60° C., are outstandingly suitable for direct oral granules. These lipids on the one hand are strong enough to form a shell around the magnesium-containing core of the extended-release particles in a stable manner and do not (begin to) melt at body temperature. In contrast to lipids having melting points of less than 50° C. or similar hydrophobic shells composed of waxes or silicones, moreover, the lipids of the invention do not leave behind a soapy or fatty (taste) impression in the mouth, and this is important for a composition in the form of direct oral granules, since these granules typically remain longer in the mouth than is the case, for example, for a peroral tablet, and are not diluted by additional liquids there.

At the same time, the lipid coatings of the invention, in comparison to conventional polymer coatings (such as those composed of cellulose ethers, for example), are perceived subjectively to be less hard and less “plasticky” in the mouth and/or on and under the tongue; hence they produce less of a “foreign body” sensation. All of this contributes significantly to the desired pleasant mouthfeel of the inventive direct oral granules.

The lipid coating may optionally comprise one or more further excipients with which its properties in terms of ease of processing, taste, appearance, stability or active ingredient release are adapted. These may be, for example, water-soluble substances, substances having a relatively low melting temperature, swellable substances and/or wetting agents such as, for example, polysorbates (e.g., Tween®). One objective of these excipients in the lipid coating—if used—is to support the stability of the film and possibly to enable more variable release profiles. Examples of excipients which are able potentially to improve the ease of processing of lipids and lipid mixtures are emulsifiers, especially those having medium or low HLB (hydrophilic-lipophilic balance) values of about 12 or less. In principle, the substances selected in this context ought of course to be only those which on the one hand do not adversely affect the taste impression and on the other hand do not have any risk of incompatibility with other coating constituents, and specifically with the lipid or lipids (substances that might lead to instances of phase separation, for example).

Preferably, however, the coating consists predominantly of one or more lipids, meaning to an extent of at least 70 wt. %. Also preferred are coatings with at least 80 wt. % lipid(s), with at least 90 wt. % lipid(s) or those which consist almost exclusively of lipid(s).

In one embodiment, the lipids in the coating of the extended-release particles are triglycerides (also called triacylglycerides, glycerol-triesters or else, less often, neutral fats), these being triple esters of glycerol with three fatty acid molecules.

In a further embodiment, the coating of the extended-release particles comprises at least 70 wt. %, or at least 80 wt. %, or at least 90 wt. % of a triglyceride. In one specific embodiment, the triglycerides in question are those of natural fatty acids, such as palmitic acid, stearic acid or arachidonic acid. In a further specific embodiment, the lipids in the coating are extremely pure forms of single triglycerides, namely triglycerides which contain at least 80 wt. % and preferably at least 90 wt. % of a single triglyceride, and only a small extent of mixed and/or partial glycerides. Examples of such single triglycerides are glycerol tripalmitate, glycerol tristearate or glycerol triarachidate. The triglycerides may optionally also be mixed with one another.

In one of the preferred embodiments, the coating of the extended-release particles comprises glycerol tripalmitate and/or glycerol tristearate, or consists essentially thereof; for example, having a coating of Dynasan®116 or 118. In one specific embodiment, the coating of the extended-release particles comprises at least 70 wt. %, or at least 80 wt. %, or at least 90 wt. % of glycerol tripalmitate and/or glycerol tristearate. In one of the preferred embodiments, the coating of the extended-release particles consists essentially of glycerol tripalmitate and/or glycerol tristearate. One of the preferences for coatings which consist to an extent of at least 90 wt. % of glycerol tripalmitate and/or glycerol tristearate, such as Dynasan®116 or 118, for example, is that they are obtained synthetically and frequently are purer than the majority of natural or semisynthetic lipids; thus, for example, they contain fewer mixed triglycerides or partial glycerides, and/or have more clearly defined melting points. This is advantageous for their processing, since, for example, a melt for spraying need not be heated to an unnecessarily high level in order fully to melt all of the constituents of the lipid.

In contrast to this, with lipids having relatively broad melting ranges it is typically necessary to operate at even above the upper value of this melting range in order to ensure a homogeneous composition of the melt and to prevent possible clogging of the spraying nozzle(s). Such ‘superheated’ melts then cure more slowly on the magnesium-containing cores receiving the spray, thus forming the desired coating less rapidly. Moreover, the agglomeration tendency may be increased due to the slower curing. Furthermore, instances of melt separation during solidification on the magnesium-containing cores that are the spraying target are possible if the higher-melting components of the melt cure first.

Furthermore, the coatings with at least 90 wt. % of glycerol tripalmitate and/or glycerol tristearate are already present in their more stable β modification after processing, and so the release typically does not change, or changes only marginally, during storage. The extended-release particles obtained in this way are therefore significantly more storage-stable.

In a further preferred embodiment, the coating of the extended-release particles consists essentially of glycerol tristearate. Glycerol tristearate (melting point Mₚ around 70-73° C.) in particular has emerged in experiments as being particularly pleasant in the mouth, owing to its higher melting point, and surprisingly it leaves behind unpleasantly soapy and/or fatty (taste) impressions to significantly less of an extent than other triglycerides having a melting point well above the body temperature, such as trilaurin (Mₚ around 44-47° C.), for example. Moreover, glycerol tristearate is significantly more taste-neutral than a host of natural or semisynthetic lipids. For example, in contrast to hydrogenated bovine tallow (Mₚ around 60° C.) or hydrogenated castor oil (Mₚ 79-80° C.), it does not leave behind any ‘coarse’ or respectively ‘raspy’ taste impression on the tongue. Even many neutral hydrogenated oils (such as rapeseed or soy) may in certain circumstances be perceived in oral granules as having an unpleasant or unfitting taste and do not go well together with fresh and/or fruity flavors such as orange, lemon, cherry, berries, tropical fruits or the like.

The release may be controlled not only via the composition of the coating but also via its thickness. The thickness in turn is a product of the application rate of the coating material and the surface area of the particles to be coated. The application rate of the coating material, with which the desired extension of release is achieved, is therefore to be selected according to the size of the magnesium-containing cores and the resultant surface area. In one of the preferred embodiments, the weight ratio between core and coating in the extended-release particles is between 60:40 and 95:5, or between 70:30 and 90:10; for example, 75:25, 80:20, 85:15 or 90:10. The specific weight ratio between core and coating may be adapted in line with the target release kinetics or extension of release and with the composition. In this context it should be borne in mind that the amount of coating material required may alter slightly even for one and the same lipid coating, if, for example, there are variations in the particle shape and size and/or in the porosity of the material for coating.

In one of the preferred embodiments, the extended-release particles are provided with a coating which consists to an extent of at least 90 wt. % of glycerol tripalmitate and/or glycerol tristearate, and in which the weight ratio between core and coating is between 60:40 and 95:5. In a likewise preferred embodiment, the extended-release particles are provided with a coating which consists to an extent of at least 90 wt. % of glycerol tristearate, or which optionally consists essentially thereof, and in which the weight ratio between core and coating is between 70:30 and 90:10, preferably between 80:20 and 90:10.

In connection with the abovementioned surface area to be coated, it should be borne in mind also that magnesium-containing cores with insufficient abrasion resistance, such as those used by the inventors in earlier hotmelt coating experiments, for example, are critical not solely because of the very labor-intensive and time-consuming working steps (clogged filters, frequent and difficult cleaning), but instead pose a problem primarily because, owing to the progressive comminution of an insufficiently stable core material in the fluidized bed, the surface area for coating changes in a barely calculable way and hence a reproducible hotmelt coating process is virtually impossible.

In vitro, the release profile can be determined in accordance with the common pharmacopeias using a standardized release apparatus under standardized conditions. In accordance with the invention, the release profile is determined preferably in a release apparatus with paddle stirrer (USP Dissolution Apparatus 2) in, for example, 900 mL of 0.1 N hydrochloric acid (HCl) at 37° C. and a stirring speed of 75 rpm. Under these conditions, the composition of the invention exhibits an extension of release over at least around 4-6 hours, preferably over at least around 6-8 hours; in other words, the active ingredient is not yet fully released before the respective period has elapsed.

In one of the preferred embodiments, the thickness of the coating in the extended-release particles is selected such that the extended-release particles over the course of 4 hours, preferably over the course of 5 hours, more preferably over the course of 6 hours release not more than 85% of the magnesium they comprise, measured in a release apparatus with paddle stirrer (USP Dissolution Apparatus 2) in 900 mL of 0.1 N hydrochloric acid at 37° C. and a stirring speed of 75 rpm. Longer extensions of release by the extended-release particles are frequently no longer purposeful, since the particles after 8-10 h are normally in the large intestine, where both the absorption surface area and the water required for release are significantly reduced.

Also preferred are versions which after 4 hours have released between 50% and 80% of the active ingredient, and/or after 6 hours between 60% and 90%, and/or after 8 hours between 65% and 100%.

In order to achieve the desired extension of release over around 6-8 h and/or of not more than 85% within 4 hours with the extended-release particles, the weight ratio between core and lipid coating ought to be between 60:40 and 90:10. Also advantageous is a weight ratio of between 80:20 and 95:5, e.g., 85:15 or 90:10. In this context, weight ratios of more than 80:20, such as 85:15, 90:10 or 95:5, for example, are also advantageous because lower application rates of the coating material customarily entail shorter process times.

While the coating of the extended-release particles primarily ensures the desired extended release of the active ingredient, it also, however, functions as a taste mask for the magnesium compound comprised in the cores, since this compound, owing to the extension of release, is released hardly or not at all on the tongue. This may be advantageous in certain circumstances specifically for certain of the magnesium compounds with a very acidic taste or a less acceptable taste, such as, for example, certain magnesium citrates or magnesium acetate.

In accordance with the invention, the composition in the form of direct oral granules is constructed such that as well as the coated magnesium extended-release particles of component (a), the granules also comprise a fraction of non-extended-release, magnesium-containing particles as component (b). These are conceived primarily as a quick-release initial dose. The amount or dose of non-extended-release magnesium is typically less than that of the coated magnesium extended-release particles; for example, about 5-100 mg of non-extended-release magnesium in combination with a dose of 50-700 mg of magnesium extended-release particles, for example around 8 mg initial dose plus 500 mg extended-release magnesium, or around 50 mg initial dose plus 350 mg extended-release magnesium. These two partial doses ought to be selected such that in total they do not exceed a daily dose of around 800 mg of magnesium, preferably around 600 mg, as far as possible.

In one embodiment, the magnesium compound in the non-extended-release particles of component (b) is selected from magnesium oxide (MgO), magnesium carbonate (MgCO₃), magnesium acetate (Mg(CH₃COO)₂), magnesium hydrogen phosphate (MgHPO₄), magnesium hydrogen citrate (C₆H₆MgO₇), trimagnesium dicitrate (C₁₂H₁₀Mg₃O₁₄), magnesium pidolate (C₁₀H₁₂MgN₂O₆), magnesium N-acetyltaurinate (C₈H₁₆MgN₂O₈S₂) and/or magnesium bis(hydrogen-L-glutamate), and also hydrates thereof. In one specific embodiment, the magnesium compound in the non-extended-release particles of component (b) is selected from magnesium hydrogen citrate and/or trimagnesium dicitrate. In a more specific embodiment, the non-extended-release particles of component (b) consist essentially of magnesium hydrogen citrate and/or trimagnesium dicitrate.

In one embodiment, the extended-release particles of component (a) comprise a different magnesium compound from the non-extended-release particles of component (b). Hence, for example, in one of the preferred embodiments, the core of the extended-release particles may comprise magnesium oxide, and the non-extended-release particles of component (b) comprise magnesium hydrogen citrate and/or trimagnesium dicitrate. Additionally preferred are embodiments in which the core of the extended-release particles consists essentially of magnesium oxide, and the non-extended-release particles of component (b) consist essentially of magnesium hydrogen citrate and/or trimagnesium dicitrate.

Trimagnesium dicitrate is a nonacid salt of citric acid, and may therefore have a more neutral taste than, for example, magnesium hydrogen citrate; in certain circumstances, this may be advantageous for direct oral granules.

In one embodiment, the extended-release particles of component (a), and optionally the non-extended-release particles of component (b), have a median particle size (D50), measured by means of dynamic image analysis (for example, using a Camsizer® XT), of ≤500 μm, preferably ≤450 μm, more preferably ≤400 μm. As mentioned above, the particle size of direct oral granules ought in general not to exceed 600 μm, preferably 500 μm, in order to prevent a “foreign body” sensation in the mouth and to keep the chewing reflex low. In other words, the majority of the particles (at least 80%, preferably at least 90%.) ought to pass through a screen having a mesh size of around 600 μm. In one preferred embodiment of the invention, therefore, the extended-release particles of component (a)—and optionally the non-extended-release particles of component (b)—have a median particle size (D50), measured by means of dynamic image analysis, of between 100 μm and 500 μm, preferably between 150 μm and 450 μm, more preferably between 200 μm and 400 μm.

In one embodiment, the extended-release particles of component (a) make up between 15 and 85 wt. %, or between 25 and 75 wt. %, or between 30 and 70 wt. %, or between 30 and 60 wt. % of the solid pharmaceutical or nutraceutical composition according to the first aspect; for example, 30-35 wt. % or 55-60 wt. %. The choice here is guided primarily by the fraction of the total magnesium dose in the composition that is to be released in an extended manner and by the level of the single dose selected for administration.

As well as the extended-release particles of component (a) and the non-extended-release, magnesium-containing particles of component (b), the composition in the form of direct oral granules comprises at least one water-soluble excipient as component (c), with which the other components are mixed. In accordance with the invention, these are excipients which, under the definition in the European Pharmacopeia, are soluble, readily soluble or very readily soluble in water (i.e., 1 g of substance for dissolution requires for its dissolution not more than 30 mL of water, or not more than 10 mL or less than 1 mL of water, respectively) and which are selected from the group of sugars, sugar alcohols and oligosaccharides. During the administration of the direct oral granules, the water-soluble excipients of component (c) act, so to speak, as the external phase of a swallowable suspension formed in situ, which allows the extended-release particles to be swallowed more easily. Preference is therefore given to selecting palatable water-soluble excipients such as sugars, sugar alcohols or water-soluble representatives of the oligosaccharides. This also assists with compliance in the case of long-term use of the oral granules. While it would theoretically be possible to administer the extended-release particles of component (a) and the magnesium-containing particles of component (b) on their own, without further excipients, they would in that case not be quite so easy and convenient to swallow, would remain in the mouth for longer, and would possibly be chewed up by the user, so undoing the extended-release effect.

The amount of excipients, especially the amount of components (c) and (d), ought preferably to be selected here such that the total amount of direct oral granules per dose is not more than around 3 g, or not more than about 2.5 g or 2.0 g, since larger amounts of powder or granules in the mouth and on the tongue are difficult to uniformly wet. Where necessary, a single dose may be taken in two or more portions (e.g., 2-3 sachets with not more than around 3 g, preferably not more than around 2 g, of oral granules).

In one embodiment, the solid pharmaceutical or nutraceutical composition according to the first aspect comprises at least 20 wt. %, preferably at least 23 wt. %, more preferably at least 25 wt. % of the excipients of component (c).

In one embodiment, the excipients of component (c) in the solid pharmaceutical or nutraceutical composition according to the first aspect comprise at least 60 wt. %, preferably at least 75 wt. %, more preferably at least 85 wt. %, of a water-soluble sugar or sugar alcohol; optionally they consist essentially thereof. In this context, the water-soluble sugar or sugar alcohol may be selected from sucrose, sorbitol, xylitol, erythritol, maltitol, isomaltitol, mannitol, and mixtures thereof. In one specific embodiment, the composition of the direct oral granules is such that the excipients of components (c) and (d) comprise at least 50 wt. %, or at least 55 wt. %, of sorbitol or xylitol, or, in the case of a mixture of sorbitol and xylitol, at least 50 wt. %, or at least 55 wt. % of this mixture. In individual embodiments, the composition of the direct oral granules may also be such that the excipients of components (c) and (d) in fact comprise at least 85 wt. %, or at least 90 wt. % of sorbitol or xylitol, or, in the case of a mixture of sorbitol and xylitol, at least 85 wt. %, or at least 90 wt. % of this mixture.

As a result of these weight fractions of the soluble sugars, sugar alcohols or oligosaccharides, a sufficient volume of highly palatable liquid suspension medium for the composition of the invention is formed in the mouth of the user and there are positive effects on swallowability and also on user compliance.

The composition in the form of direct oral granules, as well as the sugars, sugar alcohols and/or oligosaccharides, may optionally have also had further excipients under component (d) added to it, these excipients, for example, slightly raising the viscosity of the granule suspension that forms in situ on the tongue, stimulating the flow of saliva, or influencing the taste, or regulating the free-flow capacity or flowability of the granules. The excipients of (d) may optionally also be water-soluble (i.e., soluble, readily soluble or very readily soluble according to the definition in the European Pharmacopeia; i.e., 1 g of substance for dissolution requires for its dissolution not more than 30 mL of water, not more than 10 mL or less than 1 mL of water, respectively).

In one embodiment, the solid pharmaceutical or nutraceutical composition according to the first aspect comprises, for example, an acidic excipient as a constituent of the excipients of component (d). Preferred acidic excipients are citric acid, tartaric acid, malic acid, succinic acid, and also acid salts thereof, such as monosodium citrate. The inventors have determined that the use of these acidic substances—especially in combination with the soluble sugars and sugar alcohols in the preferred amounts—produces an additional stimulation of salivary flow, which provides for optimal dispersion of the composition, and especially of the customarily somewhat larger extended-release particles therein, and makes it/them particularly easy to swallow. In one specific embodiment, the composition comprises, as further excipients of component (d), citric acid, monosodium citrate and/or disodium citrate, more particularly citric acid in combination with monosodium citrate.

In a further embodiment, the composition in the form of direct oral granules may have a composition such that the excipients of component (d) comprise viscosity-increasing substances as swallowability-promoting excipients, for example, cellulose ethers such as carmellose-sodium (also called sodium carboxymethylcellulose; customarily in the non-crosslinked form) or modified starches such as, for example, Lycatab® PGS (a fully pregelatinized corn starch). Their concentration ought to be selected such that on the one hand they improve the mouthfeel of the oral granules, and stabilize the extended-release particle suspension formed from them in situ, but on the other hand they do not make swallowing more difficult; that is, the suspension formed on the tongue ought to be creamy, not slimy or sticky.

Further optionally, the direct oral granules may have a composition such that the excipients of component (d) comprise glidants: for example, flow regulators and lubricants known to the skilled person, such as finely divided silicon dioxide, magnesium stearate and/or stearic acid. These substances serve to make the direct oral granules scatterable or flowable or free-flowing, allowing them to be easily dispensed and also easily removed from packaging again. Magnesium stearate is preferred in this context but is explicitly not counted among the magnesium-containing components (a) or (b), since magnesium stearate, if used, is employed only in comparatively small amounts (≤0.50 wt. %, preferably ≤0.30 wt. %, more preferably ≤0.25 wt. %), so as to avoid a waxy impression in the mouth.

Furthermore, flavors, sweeteners, taste corrigents, colorants, etc. may be used as a constituent of the excipients of component (d) in order to adapt the organoleptic properties of the composition of the invention. Thus, the direct oral granules may, for example, have a composition such that the excipients, as well as the soluble sugars, sugar alcohols or oligosaccharides of component (c), comprise further sweeteners or sweetening agents (e.g., aspartame, acesulfame-K, sucralose, or the like), and/or flavors (lemon, orange, tropical fruits, etc.). Common and tolerated sweeteners and flavors are well known to the skilled person, from oral pediatric formulations, for example.

Further to components (a) to (d), and further in particular to the magnesium, the direct oral granules may optionally comprise further nutraceutical substances, such as minerals and/or vitamins, for example. These as well may optionally be in extended-release form—for example, having release-extending coatings and/or being embedded in a release-extending matrix. In one embodiment, the composition comprises, for example, vitamins of the B series and/or vitamin D. In one specific embodiment, the composition comprises, for example, magnesium in combination with vitamin D3 and B12. In a further specific embodiment, the composition comprises, for example, magnesium in combination with vitamin B1, B2, B6 and B12.

In one embodiment, the amounts of the extended-release particles of component (a) and of the non-extended-release particles of component (b) are selected such that within 4 hours, preferably within 5 hours, more preferably within 6 hours, they release not more than 85% of the magnesium comprised in the composition, measured in a release apparatus with paddle stirrer (USP Dissolution Apparatus 2) in 900 mL of 0.1 N hydrochloric acid at 37° C. and a stirring speed of 75 rpm. In order to ensure this, typically at least 60 wt. %, or at least 75 wt. %, or at least 85 wt. %, of the total magnesium comprised in the composition is comprised in the extended-release particles of component (a). Alternatively, or additionally to this, the composition typically comprises at least 5 wt. %, or at least 10 wt. %, or at least 12 wt. %, and up to not more than 25 wt. %, of the total magnesium comprised in the composition, in non-extended-release form.

It is optionally possible for certain or all of the substances or excipients of components (b), (c) and/or (d) that are admixed to the extended-release particles of component (a) to be present in granulated form. This may be advantageous, for example, in cases in order to reduce a potentially disruptive fine dust fraction and/or if the mixture of the extended-release particles of component (a) and the components (b), (c) and/or (d) have a tendency to separate (for example, if the particle sizes of certain or all of the substances in components (b), (c) and/or (d) are well below the particle size of the extended-release particles). A harmonization or approximation of the particle sizes of components (b), (c) and/or (d) to the particle size of the extended-release particles of component (a) through granulation may counteract these separation tendencies and so ensure uniform dispensing of the direct oral granules. In one embodiment, the admixed excipient particles of components (b), (c) and/or (d), also have a median particle size (D50), measured by means of dynamic image analysis, of between 100 μm and 500 μm, preferably between 150 μm and 450 μm, more preferably between 200 μm and 400 μm.

In one of the preferred embodiments, moreover, the composition of the direct oral granules is such that the excipients of components (c) and (d) contain not more than 5 wt. % of excipients that are sparingly soluble in water; in other words, conversely, around 95 wt. % or more of the excipients ought to be soluble. This is desirable in order to minimize the “foreign body” sensation in the mouth.

In one of the preferred embodiments, single doses of the composition are packaged in stick packs, sachets, glass ampoules or plastic ampoules, in order thus to facilitate metering of the magnesium for the user. Stick packs are particularly preferred in this context, since they can be transported easily without risk of breakage and also, when opened by tearing, typically offer a relatively small pouring aperture—and can therefore be emptied fully into the mouth more easily—than the sachets, whose construction is comparable but which are usually larger in size. Moreover, by virtue of their lamination, stick packs offer a simple possibility for protecting the oral granules from light and oxygen and so additionally stabilize the magnesium comprised in the composition.

In one embodiment, the single doses of the composition comprise typically between 50 and 700 mg of magnesium, preferably between 100 and 650 mg, more preferably between 150 and 600 mg or between 350 and 550 mg; for example, around 400 mg, around 500 mg or around 510 mg. In one specific embodiment, a single dose of the composition comprises around 350 mg of magnesium in the extended-release particles of component (a) and around 50 mg of magnesium in the non-extended-release particles of component (b); for example, around 350 mg of magnesium in the form of magnesium oxide in the extended-release particles and around 50 mg of magnesium in the form of non-extended-release magnesium hydrogen citrate, or alternatively around 350 mg of magnesium in the form of magnesium oxide mixed with around 42 mg of magnesium in the form of non-extended-release magnesium hydrogen citrate and around 8 mg of magnesium in the form of non-extended-release trimagnesium dicitrate. In a further specific embodiment, a single dose of the composition comprises around 510 mg of magnesium in the extended-release particles of component (a) and around 8 mg of magnesium in the non-extended-release particles of component (b); for example, around 500 mg of magnesium in the form of magnesium oxide and around 10 mg of magnesium in the form of non-extended-release trimagnesium dicitrate.

In one of the preferred embodiments, a composition in the form of direct oral granules is provided, comprising:
extended-release particles having a magnesium-containing core and a coating which consists to an extent of at least 90 wt. % of glycerol tripalmitate and/or glycerol tristearate, for example, Dynasan®116 or 118, the weight ratio between core and coating being between 70:30 and 90:10;
non-extended-release magnesium particles;
at least one or two or more water-soluble excipients selected from sucrose, sorbitol, xylitol, erythritol, maltitol, isomaltitol, mannitol and mixtures thereof;
at least one or two or more saliva-stimulating excipients, selected from citric acid, monosodium citrate, disodium citrate, and mixtures thereof;
optionally one or more swallowability-promoting excipients selected from cellulose ethers or modified starches and mixtures thereof;
additionally optionally a glidant selected from magnesium stearate, stearic acid and finely divided silicon dioxide or mixtures thereof;
at least one flavor; and also optionally further sweeteners or sweetening agents, the weight fraction of the extended-release particles in the direct oral granules being between 25% and 75%.

In a further preferred embodiments, a composition in the form of direct oral granules is provided, comprising:
extended-release particles having a magnesium-containing core comprising, or consisting of, magnesium oxide, magnesium carbonate or magnesium chloride, and a coating which consists to an extent of at least 90 wt. % of glycerol tripalmitate and/or glycerol tristearate, for example, Dynasan®116 or 118, the weight ratio between core and coating being between 80:20 and 90:10;
non-extended-release magnesium particles comprising, or consisting of, magnesium hydrogen citrate and/or trimagnesium dicitrate;
at least one or two or more water-soluble excipients selected from sorbitol, xylitol and mixtures thereof;
at least one or two or more saliva-stimulating excipients, selected from citric acid, monosodium citrate, and mixtures thereof;
optionally one or more swallowability-promoting excipients selected from sodium carboxymethylcellulose, fully gelatinized starch (e.g., corn starch) and mixtures thereof;
additionally optionally magnesium stearate as glidant;
at least one flavor; and also at least one sweetener selected from sucralose, aspartame, acesulfame-K, and mixtures thereof, the weight fraction of the extended-release particles in the direct oral granules being between 30% and 70%.

The advantage of the composition of the invention in the form of direct oral granules according to the first aspect of the invention is that it can typically be comprehensively wetted or suspended without additional liquid, solely by means of the saliva present in the mouth, and can subsequently be easily and conveniently swallowed, while at the same time it ensures the controlled release of the magnesium in the form of an extended-release dose in combination with a non-extended-release dose, even if in total there are relatively high doses of, for example, 400 mg of magnesium or more present. Otherwise, the latter is frequently possible only in the form of bulky capsule or tablet formulations which are difficult to swallow.

This advantageous swallowability is achieved and/or supported by factors including a finely harmonized selection and combination of parameters, such as, for example, the nature and amount of the excipients admixed to the extended-release particles, the weight fraction of the extended-release particles in the oral granules, their particle size (and also the particle size of further constituents of the oral granules), the choice of the coating materials, etc. Advantageous embodiments are described above.

The advantageous ease of processing of the extended-release dose fraction in the extended-release particles in turn is achieved by factors including the specific selection of magnesium cores having the claimed parameters, which actually enable the application of the hotmelt coating, especially on an industrial scale.

In a second aspect, the present invention relates to a method for characterizing a magnesium-containing core for producing a solid, lipid-coated pharmaceutical or nutraceutical composition in a fluidized bed (optionally the production of the extended-release particles of component (a) of the composition according to the first aspect of the invention), the core comprising or consisting of a magnesium compound, and the method being carried out by means of vibration screening, and by magnesium-containing cores which are characterized as being suitable for producing the solid, lipid-coated pharmaceutical or nutraceutical composition in a fluidized bed, and/or a sample thereof, in this test having an abrasion resistance such that after the vibration screening over a period of 60 minutes:
the fine fraction of particles having a screen diameter of <200 μm after a screening time of 60 minutes, t₆₀ₘᵢₙ, is higher by not more than 100 wt. %, preferably by not more than 50 wt. %, more preferably by not more than 20 wt. %, relative to the initial fine fraction of this screen diameter after a screening time of 5 minutes, t₅ₘᵢₙ; and/or
the fraction of particles having a screen diameter of 200 μm≤x<800 μm after a screening time of 60 minutes, t₆₀ₘᵢₙ, is lower by not more than 18 wt. %, preferably is lower by not more than 15 wt. %, more preferably is lower by not more than 12 wt. %, relative to the initial fraction of this screen diameter after a screening time of 5 minutes, t₅ₘᵢₙ.

The method according to the second aspect of the invention is therefore a suitability or serviceability test with which it is possible to examine—typically in sample form—whether, for example, a commercially available unfinished magnesium-containing core product is sufficiently abrasion-resistant to withstand the mechanical and possibly also the thermal loads of a lipid coating process, more particularly a process of hotmelt coating with lipids, in a fluidized bed.

All embodiments disclosed in conjunction with the pharmaceutical or nutraceutical composition according to the first aspect of the invention, including preferred embodiments, may be applied equally to the characterization method according to the second aspect of the invention. This relates, for example, to the observations regarding the magnesium-containing cores and also regarding the applied lipid coatings.

According to the invention, the vibration screening is carried out with a vibration amplitude of around 50/min to around 100/min, or around 70/min to around 90/min, or around 75/min to 85/min, for example, at a vibration amplitude of around 80/min.

The vibration screening in accordance with the invention described here may be carried out in principle with any screening tower which is able to ensure vibration screening at these vibration amplitudes over a period of 60 minutes, such as with a screening tower of the AS 200 series from Retsch GmbH, for example, such as the AS 200 basic model, which has been utilized primarily by the inventors.

In one embodiment, the vibration screening is carried out using a screening tower having screen trays of around 10-22 cm screen tray diameter. In a further embodiment, the vibration screening is carried out using a screening tower having screen trays of around 10-22 cm screen tray diameter and also an initial amount for testing of around 50-150 g of the magnesium-containing cores, or around 75-125 g, or around 95-105 g, for example, around 100 g of the magnesium-containing cores. In one specific embodiment, the vibration screening is carried out using a screening tower having screen trays of around 20 cm screen tray diameter, and also an initial amount for testing the magnesium-containing cores of around 100 g.

The choice of larger screen tray diameters and/or of different amounts of the magnesium-containing cores for testing than are recited here is possible in principle, without necessarily thereby departing from the spirit of the present invention. The critical factor in this case, however, is that the skilled person adapts the amount of the magnesium-containing cores for testing to the diameter of the screen trays used in the screening tower, in accordance with principles of screen analyses that are known to the skilled person, and, for example, does not overfill these trays.

In one embodiment of the method, the vibration screening is carried out using a screening aid in the form of abrasion-resistant beads having a diameter of around 2-6 mm, or around 2-5 mm, or around 2-4 mm; for example, around 3 mm. In a further embodiment of the method, the vibration screening is carried out using a screening aid in the form of abrasion-resistant beads which have a bulk density of around 650-850 kg/m³, or around 700-800 kg/m³, such as, for example, around 750 kg/m³. In one of the preferred embodiments, the vibration screening is carried out using a screening aid in the form of abrasion-resistant silica gel beads having a diameter of around 2-6 mm or around 2-5 mm, or around 2-4 mm; for example, around 3 mm.

In a further embodiment of the method, the vibration screening is carried out using a screening aid in the form of abrasion-resistant silica gel beads which have a bulk density of around 650-850 kg/m³, or around 700-800 kg/m³, for example, a bulk density of around 750 kg/m³.

In one specific embodiment of the method, the vibration screening is carried out using a screening aid in the form of abrasion-resistant silica gel beads having a diameter of around 2-6 mm, or around 2-5 mm, or around 2-4 mm (e.g., around 3 mm), and a bulk density of around 650-850 kg/m³, or around 700-800 kg/m³, for example, having a bulk density of around 750 kg/m³.

In a further embodiment of the method, the vibration screening is carried out using per screen tray around 0.5-2.0 g, or around 0.7-1.5 g, of the screening aid, for example, around 1 g. In one specific embodiment of the method, the vibration screening is carried out using per screen tray around 0.5-2.0 g, or around 0.7-1.5 g, for example around 1 g, of a screening aid in the form of abrasion-resistant silica gel beads having a diameter of around 2-6 mm, or around 2-5 mm, or around 2-4 mm (for example, around 3 mm), and a bulk density of around 650-850 kg/m³, or around 700-800 kg/m³, for example, having a bulk density of around 750 kg/m³.

In a further embodiment of the method, the vibration screening is carried out using a screening tower having screen trays of around 10-22 cm screen tray diameter, where in the screening tower the screens having a screen diameter of 800 μm, 600 μm, 500 μm, 400 μm, 300 μm, 200 μm, and 100 μm, and also the bottom cup are used.

In one specific embodiment of the method, the vibration screening is carried out using the following:
a screening tower having screens having a screen tray diameter of around 10-22 cm and screen diameters of 800 μm, 600 μm, 500 μm, 400 μm, 300 μm, 200 μm, and 100 μm, and also the bottom cup,
an initial test quantity of the magnesium-containing cores of around 50-150 g,
around 0.5-2.0 g of screening aid per screen tray,
the abrasion-resistant silica gel beads which are used as screening aid having a diameter of around 2-6 mm and a bulk density of around 650-850 kg/m³.

In a more specific embodiment of the method, the vibration screening is carried out using the following:
a screening tower having screens having a screen tray diameter of around 10-22 cm and screen diameters of 800 μm, 600 μm, 500 μm, 400 μm, 300 μm, 200 μm, and 100 μm, and also the bottom cup,
an initial test quantity of the magnesium-containing cores of around 75-125 g,
around 0.7-1.5 g of screening aid per screen tray,
the abrasion-resistant silica gel beads which are used as screening aid having a diameter of around 2-5 mm and a bulk density of around 700-800 kg/m³.

In an even more specific embodiment of the method, the vibration screening is carried out using the following:
a screening tower having screens having a screen tray diameter of around 20 cm and screen diameters of 800 μm, 600 μm, 500 μm, 400 μm, 300 μm, 200 μm, and 100 μm, and also the bottom cup,
an initial test quantity of the magnesium-containing cores of around 100 g,
around 1 g of screening aid per screen tray,
the abrasion-resistant silica gel beads which are used as screening aid having a diameter of around 2-4 mm and a bulk density of around 750 kg/m³.

In one embodiment of the method, the magnesium compound in the magnesium-containing cores is selected from magnesium oxide (MgO), magnesium carbonate (MgCO₃), magnesium chloride (MgCl₂), magnesium hydrogen phosphate (MgHPO₄) and/or magnesium acetate (Mg(CH₃COO)₂). In one specific embodiment of the method, the magnesium compound in the magnesium-containing cores is magnesium oxide (MgO).

In one embodiment of the method according to the second aspect of the invention, magnesium-containing cores which in this test have an abrasion resistance such that after the vibration screening over a period of 60 minutes:
the fine fraction of particles having a screen diameter of <200 μm after a screening time of 60 minutes, t₆₀ₘᵢₙ, is higher by not more than 100 wt. %, preferably by not more than 50 wt. %, more preferably by not more than 20 wt. %, relative to the initial fine fraction of this screen diameter after a screening time of 5 minutes, t₅ₘᵢₙ; and/or
the fraction of particles having a screen diameter of 200 μm≤x<800 μm after a screening time of 60 minutes, t₆₀ₘᵢₙ, is lower by not more than 18 wt. %, preferably by not more than 15 wt. %, more preferably by not more than 12 wt. %, relative to the initial fraction of this screen diameter after a screening time of 5 minutes, t₅ₘᵢₙ
   are suitable especially for producing the solid, lipid-coated pharmaceutical or nutraceutical composition by means of a hotmelt coating process in a fluidized bed.

In a third aspect, the present invention relates to a process for producing a solid pharmaceutical or nutraceutical composition according to the above-described first aspect of the invention, where the process contains or comprises the following steps:
(i) providing a magnesium-containing core, the core comprising or consisting of a magnesium compound;
(ii) providing a molten coating material comprising a lipid;
(iii) fluidizing the magnesium-containing cores;
(iv) spraying the fluidized magnesium-containing cores with the molten coating material;
(v) cooling the coated magnesium-containing cores, so that the lipid solidifies, and extended-release particles of component (a) are obtained which release the comprised magnesium in an extended manner; and
(vi) mixing the extended-release particles obtained in this way as component (a) with the following further components:
   (b) non-extended-release particles having a second magnesium-containing core, the particle comprising or consisting of a magnesium compound, and the particles releasing the comprised magnesium in a non-extended manner;
   (c) one or more water-soluble excipients selected from the group of sugars, sugar alcohols, and oligosaccharides; and
   (d) optionally further excipients;
wherein at least the magnesium-containing cores of the extended-release particles of component (a), and/or a sample thereof, have an abrasion resistance such that after vibration screening over a period of 60 minutes:
   the fine fraction of particles having a screen diameter of <200 μm after a screening time of 60 minutes, t₆₀ₘᵢₙ, is higher by not more than 100 wt. % relative to the initial fine fraction of this screen diameter after a screening time of 5 minutes, t₅ₘᵢₙ; and/or
   the fraction of particles having a screen diameter of 200 μm≤x<800 μm after a screening time of 60 minutes, t₆₀ₘᵢₙ, is lower by not more than 18 wt. % relative to the initial fraction of this screen diameter after a screening time of 5 minutes, t₅ₘᵢₙ.

In other words, in step (i) of the production process according to the third aspect of the invention, the only magnesium-containing cores, or batches thereof, that are provided are those which have been characterized as suitable for the production process by means of the method according to the second aspect of the invention; or the method for characterizing the magnesium-containing cores according to the second aspect of the invention may precede step (i) of the production process, so that the only magnesium-containing cores, or batches thereof, that are selected for the lipid coating process, and more particularly for a process of hotmelt coating with lipids, in a fluidized bed are those which provide the requisite abrasion resistance for this purpose, described above. In this context, however, it should be noted once again that typically only one or more samples of a batch of the magnesium-containing cores to be coated need be examined for the required abrasion parameters, and that therefore not every single production procedure according to the third aspect of the invention must also necessarily be preceded by the vibration screening characterization method according to the second aspect.

All embodiments disclosed in connection with the pharmaceutical or nutraceutical composition according to the first aspect of the invention or with the characterization method according to the second aspect of the invention, including preferred embodiments, may be applied equally to the production process according to the third aspect of the invention.

Thus, for example, in one specific embodiment, at least the magnesium-containing cores, provided in step (i), of the extended-release particles of component (a), and/or a sample thereof, have an abrasion resistance such that after vibration screening over a period of 60 minutes, the fine fraction of particles having a screen diameter of <200 μm after a screening time of 60 minutes, t₆₀ₘᵢₙ, is higher by not more than 50 wt. %, preferably by not more than 20 wt. %, relative to the initial fine fraction of this screen diameter after a screening time of 5 minutes, t₅ₘᵢₙ; and/or the fraction of particles having a screen diameter of 200 μm≤x<800 μm after a screening time of 60 minutes, t₆₀ₘᵢₙ, is lower by not more than 15 wt. %, preferably by not more than 12 wt. %, relative to the initial fraction of this screen diameter after a screening time of 5 minutes, t₅ₘᵢₙ.

In a further embodiment, at least the magnesium-containing cores, provided in step (i), of the extended-release particles of component (a), and/or a sample thereof, have an abrasion resistance such that after vibration screening over a period of 60 minutes, the fraction of particles having a screen diameter of <300 μm after a screening time of 60 minutes, t₆₀ₘᵢₙ, is higher by not more than 55 wt. %, preferably by not more than 35 wt. %, more preferably by not more than 15 wt. %, relative to the initial fraction of this screen diameter after a screening time of 5 minutes, t₅ₘᵢₙ; and/or the fraction of particles having a screen diameter of 300 μm≤x<800 μm after a screening time of 60 minutes, t₆₀ₘᵢₙ, is lower by not more than 30 wt. %, preferably by not more than 27 wt. %, more preferably by not more than 24 wt. % relative to the initial fraction of this screen diameter after a screening time of 5 minutes, t₅ₘᵢₙ.

The vibration screening may be carried out as already described above in the second aspect of the invention: an amount for screening of around 50-150 g of the magnesium-containing cores, or around 75-125 g, or around 95-105 g, for example, around 100 g, of the magnesium-containing cores is applied to the screening tower (to the topmost screen in the tower, for example, an 800 μm screen), together with around 0.5-2.0 g, or around 0.7-1.5 g, for example, around 1 g of screening aid per screen tray (here, for example, abrasion-resistant silica gel beads measuring around 2-6 mm, or around 2-5 mm, or around 2-4 mm (for example, around 3 mm) having a bulk density of around 650-850 kg/m³, or around 70-800 kg/m³, such as around 750 kg/m³, for example) and screened for 60 min at a vibration amplitude of around 50/min to around 100/min, or around 70/min to around 90/min, or around 75/min to 85/min, for example, around 80/min, and in this way mechanically stressed. In one of the advantageous embodiments the screen analysis (that is, the characterization method according to the second aspect) precedes—as described above—the process according to the third aspect of the invention, so as to examine beforehand the serviceability of the magnesium-containing cores—or, in other words, their suitability—for a lipid coating process in a fluidized bed—for example, in particular, for a hotmelt coating process in a fluidized bed.

Cores which exhibit too much abrasion (that is, more than is allowable in the context of the invention) are not suitable for hotmelt coating in a fluidized bed, especially not on the industrial scale. During earlier experiments of producing melt-coated magnesium oral granules, the inventors had determined that the severe abrasion of certain commercially available magnesium grades caused excessive clogging of the filters long before the hotmelt coating process had advanced to a sufficient point, and that this problem could not be eliminated even with the customary automated filter knockers of the fluidized bed apparatuses. The frequent filter changes, and/or filter cleaning steps, that were consequently needed in these experiments rendered the earlier processes unsuitable for the industrial scale. Moreover, as mentioned above, the resultant accumulations of encrusted abraded core material and sprayed material on the inner walls of the fluidized bed apparatus were so pronounced and solid that very time-consuming cleaning steps were always needed.

The process described in steps (i)-(v), and optionally the mixing step (vi) as well, can be carried out in fluidized bed apparatuses (known as fluid-bed coaters or air-flow-bed coaters) of any kind, provided the apparatuses allow sufficient heating in order to avoid premature solidification of the molten coating material. Temperature and spraying rate of the melt ought in any case to be adapted such as to ensure uniform formation of film on the individual core particles, since larger agglomerates, in which, for example, two or more core particles are ‘stuck’ to one another, are not conducive to a good mouthfeel of the oral granules.

As mentioned above, the coating on the extended-release particles in one of the preferred embodiments comprises a lipid having a melting point of at least 50° C., or consists essentially thereof; preferably a lipid having a melting point of at least 60° C. In order to ensure uniform application of the molten coating material, in one embodiment of the process the process air temperature in steps (iii) and/or (iv) is between 20 and 60° C., or between 20 and 50° C., or between 20 and 45° C.; for example, around 25° C. or 35° C.

In one embodiment of the process, the molten coating material provided in step (ii) is sprayed in step (iv) at a melting temperature of between 70 and 120° C., or between 75 and 110° C., or between 80 and 100° C.; for example, at around 90 to 100° C. For example, the melting point of glycerol tristearate (e.g., Dynasan®118) is around 70-73° C.; in this case, therefore, the temperature of the melt for spraying ought to be a few degrees above this—for example, at 90° C.

In one embodiment of the process, in step (iv) a spray air temperature of between 70 and 130° C., or between 75 and 125° C., or between 80 and 120° C. is used; for example, around 100° C. or 120° C. Furthermore, in one embodiment of the process in step (iv) a spraying pressure of between 0.5 and 1.5 bar is selected, or between 0.6 and 1.3 bar, or between 0.7 and 1.2 bar; for example, at 0.8 to 1.2 bar, 0.8 to 0.9 bar or 1.0 to 1.2 bar.

In one embodiment of the process, the spray amount of the molten coating material per kilogram of core material for coating in step (iv) is between 2.0 and 10.0 g/kg/min, or between 2.5 and 9.0 g/kg/min, or between 3.0 and 8.0 g/kg/min; for example, at around 3.3 g/kg/min. In one specific embodiment of the process, this process—and more particularly coating steps (iii) to (v)—is/are carried out in a fluidized bed apparatus on the laboratory scale (e.g., in a Ventilus® V-2.5), and the spray amount of the molten coating material in step (iv) is between 5.0 and 8.0 g/min, or between 5.5 and 7.5 g/min, or between 6.0 and 7.0 g/min; for example, at around 6.5 g/min.

The components (c) to (d), optionally (b) to (d), which are present according to process step (vi) in addition to the extended-release particles of component (a), are customarily first mixed homogeneously with one another, before subsequently being combined with the extended-release particles. As mentioned, optionally, certain or all of the substances or excipients of components (b), (c) and/or (d) may be granulated or aggregated with the extended-release particles prior to this combining.

In one embodiment of the process, in a further step, subsequent to step (vi), single doses of the composition are packaged into stick packs, sachets, glass ampoules or plastic ampoules.

In a further aspect of the invention, the present invention relates to a solid pharmaceutical or nutraceutical composition in the form of direct oral granules having at least dual release of active ingredient, produced by means of the process according to the third aspect of the invention.

The following list presents embodiments which are encompassed by the present invention:
1. A solid pharmaceutical or nutraceutical composition in the form of direct oral granules with at least dual release of active ingredient, comprising components as follows:
   (a) coated, extended-release particles having a magnesium-containing core and a lipid coating, the core comprising or consisting of a magnesium compound, and the extended-release particles releasing the comprised magnesium in an extended manner;
   (b) non-extended-release, magnesium-containing particles, the particles comprising or consisting of a magnesium compound, and the particles releasing the comprised magnesium in a non-extended manner;
   (c) one or more water-soluble excipients selected from the group of sugars, sugar alcohols, and oligosaccharides; and
   (d) optionally one or more further excipients;
   wherein at least the magnesium-containing cores of the extended-release particles of component (a), and/or a sample thereof, have an abrasion resistance such that after vibration screening over a period of 60 minutes:
      the fine fraction of particles having a screen diameter of <200 μm after a screening time of 60 minutes, t₆₀ₘᵢₙ, is higher by not more than 100 wt. % relative to the initial fine fraction of this screen diameter after a screening time of 5 minutes, t₅ₘᵢₙ; and/or
      the fraction of particles having a screen diameter of 200 μm≤x<800 μm after a screening time of 60 minutes, t₆₀ₘᵢₙ, is lower by not more than 18 wt. % relative to the initial fraction of this screen diameter after a screening time of 5 minutes, t₅ₘᵢₙ
2. The composition as in embodiment 1, wherein at least the magnesium-containing cores of the extended-release particles of component (a), and/or a sample thereof, have an abrasion resistance such that after vibration screening over a period of 60 minutes:
   the fine fraction of particles having a screen diameter of <200 μm after a screening time of 60 minutes, t₆₀ₘᵢₙ, is higher by not more than 50 wt. % relative to the initial fine fraction of this screen diameter after a screening time of 5 minutes, t₅ₘᵢₙ; and/or
   the fraction of particles having a screen diameter of 200 μm≤x<800 μm after a screening time of 60 minutes, t₆₀ₘᵢₙ, is lower by not more than 15 wt. % relative to the initial fraction of this screen diameter after a screening time of 5 minutes, t₅ₘᵢₙ
3. The composition as in embodiments 1 to 2, wherein at least the magnesium-containing cores of the extended-release particles of component (a), and/or a sample thereof, have an abrasion resistance such that after vibration screening over a period of 60 minutes:
   the fine fraction of particles having a screen diameter of <200 μm after a screening time of 60 minutes, t₆₀ₘᵢₙ, is higher by not more than 20 wt. % relative to the initial fine fraction of this screen diameter after a screening time of 5 minutes, t₅ₘᵢₙ; and/or
   the fraction of particles having a screen diameter of 200 μm≤x<800 μm after a screening time of 60 minutes, t₆₀ₘᵢₙ, is lower by not more than 12 wt. % relative to the initial fraction of this screen diameter after a screening time of 5 minutes, t₅ₘᵢₙ
4. The composition as in any of the preceding embodiments, wherein at least the magnesium-containing cores of the extended-release particles of component (a), and/or a sample thereof, have an abrasion resistance such that after vibration screening over a period of 60 minutes:
   the fraction of particles having a screen diameter of <300 μm after a screening time of 60 minutes, t₆₀ₘᵢₙ, is higher by not more than 55 wt. % relative to the initial fraction of this screen diameter after a screening time of 5 minutes, t₅ₘᵢₙ; and/or
   the fraction of particles having a screen diameter of 300 μm≤x<800 μm after a screening time of 60 minutes, t₆₀ₘᵢₙ, is lower by not more than 30 wt. % relative to the initial fraction of this screen diameter after a screening time of 5 minutes, t₅ₘᵢₙ
5. The composition as in any of the preceding embodiments, wherein at least the magnesium-containing cores of the extended-release particles of component (a), and/or a sample thereof, have an abrasion resistance such that after vibration screening over a period of 60 minutes:
   the fraction of particles having a screen diameter of <300 μm after a screening time of 60 minutes, t₆₀ₘᵢₙ, is higher by not more than 35 wt. % relative to the initial fraction of this screen diameter after a screening time of 5 minutes, t₅ₘᵢₙ; and/or
   the fraction of particles having a screen diameter of 300 μm≤x<800 μm after a screening time of 60 minutes, t₆₀ₘᵢₙ, is lower by not more than 27 wt. % relative to the initial fraction of this screen diameter after a screening time of 5 minutes, t₅ₘᵢₙ
6. The composition as in any of the preceding embodiments, wherein at least the magnesium-containing cores of the extended-release particles of component (a), and/or a sample thereof, have an abrasion resistance such that after vibration screening over a period of 60 minutes:
   the fraction of particles having a screen diameter of <300 μm after a screening time of 60 minutes, t₆₀ₘᵢₙ, is higher by not more than 15 wt. % relative to the initial fraction of this screen diameter after a screening time of 5 minutes, t₅ₘᵢₙ; and/or
   the fraction of particles having a screen diameter of 300 μm≤x<800 μm after a screening time of 60 minutes, t₆₀ₘᵢₙ, is lower by not more than 24 wt. % relative to the initial fraction after a screening time of 5 minutes, t₅ₘᵢₙ.
7. The composition as in any of the preceding embodiments, wherein the vibration screening is carried out according to the characterization method as in embodiments 47 to 66, for example, with 100 g of the magnesium-containing cores and with 1 g of screening aid per screen tray and at a vibration amplitude of 80/min.
8. The composition as in embodiment 7, wherein the screening aid is in the form of abrasion-resistant silica gel beads which have a diameter of around 2-6 mm, or around 2-5 mm, or around 2-4 mm (e.g., around 3 mm) and a bulk density of around 650-850 kg/m³, or around 700-800 kg/m³, e.g., approximately 750 kg/m³.
9. The composition as in any of the preceding embodiments, wherein the magnesium-containing cores of the extended-release particles are produced by means of dry compacting.
10. The composition as in any of the preceding embodiments, wherein the magnesium-containing cores of the extended-release particles have a median particle size (D50), measured by means of dynamic image analysis (using, e.g., Camsizer® XT, Retsch Technology GmbH, Haan, Germany), of between 150 μm and 300 μm, preferably between 165 μm and 285 μm, more preferably between 180 μm and 270 μm.
11. The composition as in any of the preceding embodiments, wherein the magnesium-containing cores of the extended-release particles have a unimodal particle size distribution.
12. The composition as in any of the preceding embodiments, wherein the magnesium-containing cores of the extended-release particles have a particle size distribution whose narrowness is such that the quotient (D90-D10)/D50 is less than 1.60, preferably less than 1.50, and more preferably less than 1.40.
13. The composition as in any of the preceding embodiments, wherein at least the magnesium compound in the extended-release particles of component (a) in its anhydrous form has a magnesium content of 15 wt. % or more, preferably 25 wt. % or more, more preferably 50 wt. % or more.
14. The composition as in any of the preceding embodiments, wherein the magnesium compound in the extended-release particles is selected from magnesium oxide (MgO), magnesium carbonate (MgCO₃), magnesium chloride (MgCl₂), magnesium hydrogen phosphate (MgHPO₄) and/or magnesium acetate (Mg(CH₃COO)₂).
15. The composition as in any of the preceding embodiments, wherein the core of the extended-release particles optionally comprises one or more excipients.
16. The composition as in any of the preceding embodiments, wherein the magnesium compound in the core of the extended-release particles is magnesium oxide (MgO).
17. The composition as in any of the preceding embodiments, wherein the core in the extended-release particles consists substantially of magnesium oxide (MgO).
18. The composition as in any of the preceding embodiments, wherein the coating of the extended-release particles comprises, or substantially consists of, a lipid having a melting point of at least 50° C., preferably a lipid having a melting point of at least 60° C.
19. The composition as in any of the preceding embodiments, wherein the lipids in the coating of the extended-release particles comprise at least one triglyceride.
20. The composition as in any of the preceding embodiments, wherein the coating of the extended-release particles comprises at least 70 wt. %, or at least 80 wt. %, or at least 90 wt. % of a triglyceride.
21. The composition as in any of embodiments 19 or 20, wherein the triglyceride comprises natural fatty acids.
22. The composition as in any of the preceding embodiments, wherein the coating of the extended-release particles comprises, or substantially consists of, glycerol tripalmitate and/or glycerol tristearate.
23. The composition as in any of the preceding embodiments, wherein the coating of the extended-release particles comprises at least 70 wt. %, or at least 80 wt. %, or at least 90 wt. % of glycerol tripalmitate and/or glycerol tristearate.
24. The composition as in any of the preceding embodiments, wherein the coating of the extended-release particles consists essentially of glycerol tripalmitate and/or glycerol tristearate.
25. The composition as in any of the preceding embodiments, wherein the coating of the extended-release particles consists essentially of glycerol tristearate.
26. The composition as in any of the preceding embodiments, wherein the weight ratio between core and coating in the extended-release particles is between 60:40 and 95:5, or between 70:30 and 90:10.
27. The composition as in any of the preceding embodiments, wherein the strength of the coating in the extended-release particles is selected such that the extended-release particles over the course of 4 hours, preferably over the course of 5 hours, more preferably over the course of 6 hours, release not more than 80% of the magnesium they comprise, measured in a release apparatus with paddle stirrer (USP Dissolution Apparatus 2) in 900 mL of 0.1 N hydrochloric acid at 37° C. and a stirring speed of 75 rpm.
28. The composition as in any of the preceding embodiments, wherein the magnesium compound in the non-extended-release particles of component (b) is selected from magnesium oxide (MgO), magnesium carbonate (MgCO₃), magnesium acetate (Mg(CH₃COO)₂), magnesium hydrogen phosphate (MgHPO₄), magnesium hydrogen citrate (C₆H₆MgO₇), trimagnesium dicitrate (C₁₂H₁₀Mg₃O₁₄), magnesium pidolate (C₁₀H₁₂MgN₂O₆), magnesium N-acetyltaurinate (C₈H₁₆MgN₂O₈S₂) and/or magnesium bis(hydrogen-L-glutamate), and also their hydrates.
29. The composition as in any of the preceding embodiments, wherein the magnesium compound in the non-extended-release particles of component (b) is selected from magnesium hydrogen citrate and/or trimagnesium dicitrate.
30. The composition as in any of the preceding embodiments, wherein the non-extended-release particles of component (b) consist essentially of magnesium hydrogen citrate and/or trimagnesium dicitrate.
31. The composition as in any of the preceding embodiments, wherein the extended-release particles of component (a) comprise a different magnesium compound from the non-extended-release particles of component (b).
32. The composition as in embodiment 31, wherein the core of the extended-release particles comprises magnesium oxide (MgO), and the non-extended-release particles of component (b) comprise magnesium hydrogen citrate and/or trimagnesium dicitrate.
33. The composition as in embodiment 32, wherein the core of the extended-release particles consists essentially of magnesium oxide (MgO), and the non-extended-release particles of component (b) consist essentially of magnesium hydrogen citrate and/or trimagnesium dicitrate.
34. The composition as in any of the preceding embodiments, wherein the extended-release particles of component (a), and optionally the non-extended-release particles of component (b), have a median particle size (D50), measured by means of dynamic image analysis (using, e.g., Camsizer® XT, Retsch Technology GmbH, Haan, Germany), of 500 μm, preferably 450 μm, more preferably 400 μm.
35. The composition as in embodiment 34, wherein the extended-release particles of component (a), and optionally the non-extended-release particles of component (b), have a median particle size (D50), measured by means of dynamic image analysis, of between 100 μm and 500 μm, preferably between 150 μm and 450 μm, more preferably between 200 μm and 400 μm.
36. The composition as in any of the preceding embodiments, wherein the extended-release particles of component (a) make up between 15 and 85 wt. %, or between 25 and 75 wt. %, or between 30 and 70 wt. %, or between 30 and 60 wt. % of the composition.
37. The composition as in any of the preceding embodiments, which comprises at least 20 wt. %, preferably at least 23 wt. %, more preferably at least 25 wt. % of the excipients of component (c).
38. The composition as in any of the preceding embodiments, wherein the excipients of component (c) comprise, or optionally consist of, at least 60 wt. %, preferably at least 75 wt. %, more preferably at least 85 wt. %, of a water-soluble sugar or sugar alcohol.
39. The composition as in embodiment 38, wherein the water-soluble sugar or sugar alcohol is selected from sucrose, sorbitol, xylitol, erythritol, maltitol, isomaltitol, mannitol, and mixtures thereof
40. The composition as in any of the preceding embodiments, wherein the composition comprises as further excipients of component (d) citric acid, monosodium citrate and/or disodium citrate, more particularly citric acid in combination with monosodium citrate.
41. The composition as in any of the preceding embodiments, wherein the amounts of the extended-release particles of component (a) and of the non-extended-release particles of component (b) are selected such that over the course of 4 hours, preferably over the course of 5 hours, more preferably over the course of 6 hours, release not more than 80% of the magnesium comprised in the composition, measured in a release apparatus with paddle stirrer (USP Dissolution Apparatus 2) in 900 mL of 0.1 N hydrochloric acid at 37° C. and a stirring speed of 75 rpm.
42. The composition as in any of the preceding embodiments, wherein at least 60 wt. %, or at least 75 wt. %, or at least 85 wt. %, of the total magnesium comprised in the composition is comprised in the extended-release particles of component (a).
43. The composition as in any of the preceding embodiments, wherein the composition comprises at least 5 wt. %, or at least 10 wt. %, or at least 12 wt. % of the total magnesium comprised in the composition in non-extended-release form.
44. The composition as in any of the preceding embodiments, wherein the components of (b), (c) and/or (d) are present optionally in granulated form.
45. The composition as in any of the preceding embodiments, wherein the excipients of components (c) and (d) comprise not more than 5 wt. % of excipients sparingly soluble in water.
46. The composition as in any of the preceding embodiments, wherein single doses of the composition are packaged in stick packs, sachets, glass ampoules or plastic ampoules.
47. A method for characterizing a magnesium-containing core for producing a solid, lipid-coated pharmaceutical or nutraceutical composition in a fluidized bed (optionally the production of the extended-release particles of component (a) of the composition as in embodiments 1 to 46), the core comprising or consisting of a magnesium compound, and wherein this method comprises being carried out by means of vibration screening and magnesium-containing cores which are characterized as being suitable for producing the solid, lipid-coated pharmaceutical or nutraceutical composition in a fluidized bed in this test having an abrasion resistance such that after the vibration screening over a period of 60 minutes:
   the fine fraction of particles having a screen diameter of <200 μm after a screening time of 60 minutes, t₆₀ₘᵢₙ, is higher by not more than 100 wt. %, preferably by not more than 50 wt. %, more preferably by not more than 20 wt. %, relative to the initial fine fraction of this screen diameter after a screening time of 5 minutes, t₅ₘᵢₙ; and/or
   the fraction of particles having a screen diameter of 200 μm≤x<800 μm after a screening time of 60 minutes, t₆₀ₘᵢₙ, is lower by not more than 18 wt. %, preferably by not more than 15 wt. %, more preferably by not more than 12 wt. %, relative to the initial fraction of this screen diameter after a screening time of 5 minutes, t₅ₘᵢₙ
48. The method as in embodiment 47, wherein the vibration screening is carried out at a vibration amplitude of around 50/min to around 100/min, or around 70/min to around 90/min, or around 75/min to 85/min, for example, at a vibration amplitude of around 80/min.
49. The method as in embodiment 47 or 48, wherein a screening tower having screen trays of around 10-22 cm screen tray diameter is used for the vibration screening.
50. The method as in one of the preceding embodiments, wherein a screening tower having screen trays of around 10-22 cm screen tray diameter is used for the vibration screening and also an initial test quantity of around 50-150 g of the magnesium-containing cores, or around 75-125 g, or around 95-105 g, for example, around 100 g of the magnesium-containing cores.
51. The method as in one of the preceding embodiments, wherein a screening tower having screen trays of around 20 cm screen tray diameter is used for the vibration screening and also an initial test quantity of the magnesium-containing cores of around 100 g.
52. The method as in any of the preceding embodiments, wherein a screening aid in the form of abrasion-resistant beads having a diameter of around 2-6 mm, or around 2-5 mm, or around 2-4 mm, for example, around 3 mm, is used for the vibration screening.
53. The method as in any of the preceding embodiments, wherein a screening aid in the form of abrasion-resistant beads which have a bulk density of around 650-850 kg/m³, for example, a bulk density of around 700-800 kg/m³ such as, for example, around 750 kg/m³, is used for the vibration screening.
54. The method as in any of the preceding embodiments, wherein a screening aid in the form of abrasion-resistant silica gel beads is used for the vibration screening.
55. The method as in any of the preceding embodiments, wherein a screening aid in the form of abrasion-resistant silica gel beads having a diameter of around 2-6 mm, or around 2-5 mm, or around 2-4 mm, for example, around 3 mm is used.
56. The method as in any of the preceding embodiments, wherein a screening aid in the form of abrasion-resistant silica gel beads having a bulk density of around 650-850 kg/m³, or around 700-800 kg/m³, for example, having a bulk density of around 750 kg/m³, is used.
57. The method as in any of preceding embodiments 55 or 56, wherein a screening aid in the form of abrasion-resistant silica gel beads having a diameter of around 2-6 mm, or around 2-5 mm, or around 2-4 mm (e.g. 3 mm), and having a bulk density of around 650-850 kg/m³, or around 700-800 kg/m³, for example having a bulk density of around 750 kg/m³, is used.
58. The method as in any of preceding embodiments 55 to 57, wherein per screen tray around 0.5-2.0 g, or around 0.7-1.5 g, for example, around 1 g of the screening aid, is used.
59. The method as in any of preceding embodiments 55 to 58, wherein per screen tray around 0.5-2.0 g, or around 0.7-1.5 g, for example, around 1 g of a screening aid in the form of abrasion-resistant silica gel beads having a diameter of around 2-6 mm, or around 2-5 mm, or around 2-4 mm (e.g. 3 mm), and having a bulk density of around 650-850 kg/m³, or around 700-800 kg/m³, for example, having a bulk density of around 750 kg/m³, is used.
60. The method as in any of the preceding embodiments, wherein the vibration screening is carried out using a screening tower having screen trays of around 10-22 cm screen tray diameter, and wherein in the screening tower the screens having a screen diameter of 800 μm, 600 μm, 500 μm, 400 μm, 300 μm, 200 μm, and 100 μm and also the bottom cup are used.
61. The method as in any of the preceding embodiments, wherein the following are used for the vibration screening:
   a screening tower having screens having a screen tray diameter of around 10-22 cm and screen diameters of 800 μm, 600 μm, 500 μm, 400 μm, 300 μm, 200 μm, and 100 μm, and also the bottom cup,
   an initial test quantity of the magnesium-containing cores of around 50-150 g,
   around 0.5-2.0 g of screening aid per screen tray,
   the abrasion-resistant silica gel beads which are used as screening aid having a diameter of around 2-6 mm and a bulk density of around 650-850 kg/m³.
62. The method as in any of the preceding embodiments, wherein the following are used for the vibration screening:
   a screening tower having screens having a screen tray diameter of around 10-22 cm and screen diameters of 800 μm, 600 μm, 500 μm, 400 μm, 300 μm, 200 μm, and 100 μm, and also the bottom cup,
   an initial test quantity of the magnesium-containing cores of around 75-125 g,
   around 0.7-1.5 g of screening aid per screen tray,
   the abrasion-resistant silica gel beads which are used as screening aid having a diameter of around 2-5 mm and a bulk density of around 700-800 kg/m³.
63. The method as in any of the preceding embodiments, wherein the following are used for the vibration screening:
   a screening tower having screens having a screen tray diameter of around 20 cm and screen diameters of 800 μm, 600 μm, 500 μm, 400 μm, 300 μm, 200 μm, and 100 μm, and also the bottom cup,
   an initial test quantity of the magnesium-containing cores of around 100 g,
   around 1 g of screening aid per screen tray,
   the abrasion-resistant silica gel beads which are used as screening aid having a diameter of around 2-4 mm and a bulk density of around 750 kg/m³.
64. The method as in any of the preceding embodiments, wherein the magnesium compound in the magnesium-containing cores is selected from magnesium oxide (MgO), magnesium carbonate (MgCO₃), magnesium chloride (MgCl₂), magnesium hydrogen phosphate (MgHPO₄) and/or magnesium acetate (Mg(CH₃COO)₂).
65. The method as in any of the preceding embodiments, wherein the magnesium compound in the magnesium-containing cores is magnesium oxide (MgO).
66. The method as in any of the preceding embodiments, wherein magnesium-containing cores which in this test have an abrasion resistance such that after the vibration screening over a period of 60 minutes:
   the fine fraction of particles having a screen diameter of <200 μm after a screening time of 60 minutes, t₆₀ₘᵢₙ, is higher by not more than 100 wt. %, preferably by not more than 50 wt %, more preferably by not more than 20 wt. %, relative to the initial fine fraction of this screen diameter after a screening time of 5 minutes, t₅ₘᵢₙ; and/or
   the fraction of particles having a screen diameter of 200 μm≤x<800 μm after a screening time of 60 minutes, t₆₀ₘᵢₙ, is lower by not more than 18 wt. %, preferably by not more than 15 wt. %, more preferably by not more than 12 wt. %, relative to the initial fraction of this screen diameter after a screening time of 5 minutes, t₅ₘᵢₙ
   are suitable especially for producing the solid, lipid-coated pharmaceutical or nutraceutical composition by means of a hotmelt coating process in a fluidized bed.
67. A process for producing a solid pharmaceutical or nutraceutical composition as in embodiments 1 to 46, comprising the following steps:
   (i) providing a magnesium-containing core, the core comprising or consisting of a magnesium compound;
   (ii) providing a molten coating material comprising a lipid;
   (iii) fluidizing the magnesium-containing cores;
   (iv) spraying the fluidized magnesium-containing cores with the molten coating material;
   (v) cooling the coated magnesium-containing cores, so that the lipid solidifies and extended-release particles of component (a) are obtained which release the comprised magnesium in an extended manner; and
   (vi) mixing the extended-release particles obtained in this way as component (a) with the following further components:
      (b) non-extended-release particles having a second magnesium-containing core, the particle comprising or consisting of a magnesium compound, and the particles releasing the comprised magnesium in a non-extended manner;
      (c) one or more water-soluble excipients selected from the group of sugars, sugar alcohols, and oligosaccharides; and
      (d) optionally further excipients;
   wherein at least the magnesium-containing cores of the extended-release particles of component (a), and/or a sample thereof, have an abrasion resistance such that after vibration screening over a period of 60 minutes:
      the fine fraction of particles having a screen diameter of <200 μm after a screening time of 60 minutes, t₆₀ₘᵢₙ, is higher by not more than 100 wt. % relative to the initial fine fraction of this screen diameter after a screening time of 5 minutes, t₅ₘᵢₙ; and/or
      the fraction of particles having a screen diameter of 200 μm≤x<800 μm after a screening time of 60 minutes, t₆₀ₘᵢₙ, is lower by not more than 18 wt. % relative to the initial fraction of this screen diameter after a screening time of 5 minutes, t₅ₘᵢₙ.
68. The process as in embodiment 67, wherein the characterization method as in embodiments 47 to 66 precedes step (i), or wherein the characterization method as in embodiments 47 to 66 precedes step (i) at least for a sample of the magnesium-containing cores provided in step (i).
69. The process as in embodiments 67 or 68, wherein at least the magnesium-containing cores, provided in step (i), of the extended-release particles of component (a), and/or a sample thereof, have an abrasion resistance such that after vibration screening over a period of 60 minutes:
   the fine fraction of particles having a screen diameter of <200 μm after a screening time of 60 minutes, t₆₀ₘᵢₙ, is higher by not more than 50 wt. % relative to the initial fine fraction of this screen diameter after a screening time of 5 minutes, t₅ₘᵢₙ; and/or
   the fraction of particles having a screen diameter of 200 μm≤x<800 μm after a screening time of 60 minutes, t₆₀ₘᵢₙ, is lower by not more than 15 wt. % relative to the initial fraction of this screen diameter after a screening time of 5 minutes, t₅ₘᵢₙ.
70. The process as in embodiments 67 to 69, wherein at least the magnesium-containing cores, provided in step (i), of the extended-release particles of component (a), and/or a sample thereof, have an abrasion resistance such that after vibration screening over a period of 60 minutes:
   the fine fraction of particles having a screen diameter of <200 μm after a screening time of 60 minutes, t₆₀ₘᵢₙ, is higher by not more than 20 wt. % relative to the initial fine fraction of this screen diameter after a screening time of 5 minutes, t₅ₘᵢₙ; and/or
   the fraction of particles having a screen diameter of 200 μm≤x<800 μm after a screening time of 60 minutes, t₆₀ₘᵢₙ, is lower by not more than 12 wt. % relative to the initial fraction of this screen diameter after a screening time of 5 minutes, t₅ₘᵢₙ.
71. The process as in any of the preceding embodiments, wherein at least the magnesium-containing cores, provided in step (i), of the extended-release particles of component (a), and/or a sample thereof, have an abrasion resistance such that after vibration screening over a period of 60 minutes:
   the fraction of particles having a screen diameter of <300 μm after a screening time of 60 minutes, t₆₀ₘᵢₙ, is higher by not more than 55 wt. % relative to the initial fraction of this screen diameter after a screening time of 5 minutes, t₅ₘᵢₙ; and/or
   the fraction of particles having a screen diameter of 300 μm≤x<800 μm after a screening time of 60 minutes, t₆₀ₘᵢₙ, is lower by not more than 30 wt. % relative to the initial fraction of this screen diameter after a screening time of 5 minutes, t₅ₘᵢₙ.
72. The process as in any of the preceding embodiments, wherein at least the magnesium-containing cores, provided in step (i), of the extended-release particles of component (a), and/or a sample thereof, have an abrasion resistance such that after vibration screening over a period of 60 minutes:
   the fraction of particles having a screen diameter of <300 μm after a screening time of 60 minutes, t₆₀ₘᵢₙ, is higher by not more than 35 wt. % relative to the initial fraction of this screen diameter after a screening time of 5 minutes, t₅ₘᵢₙ; and/or
   the fraction of particles having a screen diameter of 300 μm≤x<800 μm after a screening time of 60 minutes, t₆₀ₘᵢₙ, is lower by not more than 27 wt. % relative to the initial fraction of this screen diameter after a screening time of 5 minutes, t₅ₘᵢₙ.
73. The process as in any of the preceding embodiments, wherein at least the magnesium-containing cores, provided in step (i), of the extended-release particles of component (a), and/or a sample thereof, have an abrasion resistance such that after vibration screening over a period of 60 minutes:
   the fraction of particles having a screen diameter of <300 μm after a screening time of 60 minutes, t₆₀ₘᵢₙ, is higher by not more than 15 wt. % relative to the initial fraction of this screen diameter after a screening time of 5 minutes, t₅ₘᵢₙ; and/or
   the fraction of particles having a screen diameter of 300 μm≤x<800 μm after a screening time of 60 minutes, t₆₀ₘᵢₙ, is lower by not more than 24 wt. % relative to the initial fraction after a screening time of 5 minutes, t₅ₘᵢₙ
74. The process as in any of the preceding embodiments, wherein the vibration screening is carried out according to the characterization method of embodiments 47 to 66, for example, with 100 g of the magnesium-containing cores and with 1 g of screening aid per screen tray and at a vibration amplitude of 80/min.
75. The composition as in embodiment 74, wherein the screening aid is in the form of abrasion-resistant silica gel beads which have a diameter of around 2-6 mm, or around 2-5 mm, or around 2-4 mm (e.g., around 3 mm) and a bulk density of around 650-850 kg/m³, or around 700-800 kg/m³, e.g., approximately 750 kg/m³.
76. The process as in any of the preceding embodiments, wherein the magnesium-containing cores, provided in step (i), of the extended-release particles are produced by means of dry compacting.
77. The process as in any of the preceding embodiments, wherein the magnesium-containing cores, provided in step (i), of the extended-release particles have a median particle size (D50), measured by means of dynamic image analysis (using, e.g., Camsizer® XT, Retsch Technology GmbH, Haan, Germany), of between 150 μm and 300 μm, preferably between 165 μm and 285 μm, more preferably between 180 μm and 270 μm.
78. The process as in any of the preceding embodiments, wherein the magnesium-containing cores, provided in step (i), of the extended-release particles have a unimodal particle size distribution.
79. The process as in any of the preceding embodiments, wherein the magnesium-containing cores, provided in step (i), of the extended-release particles have a particle size distribution whose narrowness is such that the quotient (D90-D10)/D50 is less than 1.60, preferably less than 1.50, and more preferably less than 1.40.
80. The process as in any of the preceding embodiments, wherein the extended-release particles, provided in step (i), of component (a) comprise a different magnesium compound from the non-extended-release particles of component (b) that are admixed in step (vi).
81. The process as in any of the preceding embodiments, wherein at least the magnesium compound in the extended-release particles of component (a) in its anhydrous form has a magnesium content of 15 wt. % or more, preferably 25 wt. % or more, more preferably 50 wt. % or more.
82. The process as in any of the preceding embodiments, wherein the magnesium compound in the extended-release particles is selected from magnesium oxide (MgO), magnesium carbonate (MgCO₃), magnesium chloride (MgCl₂), magnesium hydrogen phosphate (MgHPO₄) and/or magnesium acetate (Mg(CH₃COO)₂).
83. The process as in any of the preceding embodiments, wherein the core of the extended-release particles optionally comprises one or more excipients.
84. The process as in any of the preceding embodiments, wherein the magnesium compound in the core of the extended-release particles is magnesium oxide (MgO).
85. The process as in any of the preceding embodiments, wherein the core in the extended-release particles consists substantially of magnesium oxide (MgO).
86. The process as in any of the preceding embodiments, wherein the coating material, provided in step (ii), of the extended-release particles comprises, or consists essentially of, a lipid having a melting point of at least 50° C., preferably a lipid having a melting point of at least 60° C.
87. The process as in any of the preceding embodiments, wherein the lipids in the coating material of the extended-release particles comprise at least one triglyceride.
88. The process as in any of the preceding embodiments, wherein the coating material, provided in step (ii), of the extended-release particles comprises at least 70 wt. %, or at least 80 wt. %, or at least 90 wt. % of a triglyceride.
89. The process as in any of embodiments 87 or 88, wherein the triglyceride comprises natural fatty acids.
90. The process as in any of the preceding embodiments, wherein the coating material, provided in step (ii), of the extended-release particles comprises, or consists essentially of, glycerol tripalmitate and/or glycerol tristearate.
91. The process as in any of the preceding embodiments, wherein the coating material, provided in step (ii), of the extended-release particles comprises at least 70 wt. %, or at least 80 wt. %, or at least 90 wt. % of glycerol tripalmitate and/or glycerol tristearate.
92. The process as in any of the preceding embodiments, wherein the coating material, provided in step (ii), of the extended-release particles consists essentially of glycerol tripalmitate and/or glycerol tristearate.
93. The process as in any of the preceding embodiments, wherein the coating material, provided in step (ii), of the extended-release particles consists essentially of glycerol tristearate.
94. The process as in any of the preceding embodiments, wherein in step (iv) the weight ratio between the fluidized magnesium-containing cores and the molten coating material is between 60:40 and 95:5, or between 70:30 and 90:10.
95. The process as in any of the preceding embodiments, wherein the strength of the coating applied in steps (iv) and (v) is selected such that the extended-release particles over the course of 4 hours, preferably over the course of 5 hours, more preferably over the course of 6 hours, release not more than 80% of the magnesium they comprise, measured in a release apparatus with paddle stirrer (USP Dissolution Apparatus 2) in 900 mL of 0.1 N hydrochloric acid at 37° C. and a stirring speed of 75 rpm.
96. The process as in any of the preceding embodiments, wherein the magnesium compound in the non-extended-release particles of component (b) that are admixed in step (vi) is selected from magnesium oxide (MgO), magnesium carbonate (MgCO₃), magnesium acetate (Mg(CH₃COO)₂), magnesium hydrogen phosphate (MgHPO₄), magnesium hydrogen citrate (C₆H₆MgO₇), trimagnesium dicitrate (C₁₂H₁₀Mg₃O₁₄), magnesium pidolate (C₁₀H₁₂MgN₂O₆), magnesium N-acetyltaurinate (C₈H₁₆MgN₂O₈S₂) and/or magnesium bis(hydrogen-L-glutamate), and also their hydrates.
97. The process as in any of the preceding embodiments, wherein the magnesium compound in the non-extended-release particles of component (b) that are admixed in step (vi) is selected from magnesium hydrogen citrate and/or trimagnesium dicitrate.
98. The process as in any of the preceding embodiments, wherein the non-extended-release particles of component (b) that are admixed in step (vi) consist essentially of magnesium hydrogen citrate and/or trimagnesium dicitrate.
99. The process as in any of the preceding embodiments, wherein the extended-release particles of component (a) comprise a different magnesium compound from the non-extended-release particles of component (b) that are admixed in step (vi).
100. The composition as in embodiment 99, wherein the core of the extended-release particles comprises magnesium oxide (MgO), and the non-extended-release particles of component (b) that are admixed in step (vi) comprise magnesium hydrogen citrate and/or trimagnesium dicitrate.
101. The composition as in embodiment 100, wherein the core of the extended-release particles consists essentially of magnesium oxide (MgO), and the non-extended-release particles of component (b) that are admixed in step (vi) consist essentially of magnesium hydrogen citrate and/or trimagnesium dicitrate.
102. The process as in any of the preceding embodiments, wherein the extended-release particles of component (a), and optionally the non-extended-release particles of component (b), that are admixed in step (vi) have a median particle size (D50), measured by means of dynamic image analysis (using, e.g., Camsizer® XT, Retsch Technology GmbH, Haan, Germany), of 500 μm, preferably 450 μm, more preferably 400 μm.
103. The process as in embodiment 102, wherein the extended-release particles of component (a), and optionally the non-extended-release particles of component (b), have a median particle size (D50), measured by means of dynamic image analysis, of between 100 μm and 500 μm, preferably between 150 μm and 450 μm, more preferably between 200 μm and 400 μm.
104. The process as in any of the preceding embodiments, wherein in step (vi) the extended-release particles of component (a) make up between 15 and 85 wt. %, or between 25 and 75 wt. %, or between 30 and 70 wt. %, or between 30 and 60 wt. % of the composition.
105. The process as in any of the preceding embodiments, wherein in step (vi) the excipients of component (c) make up at least 20 wt. %, preferably at least 23 wt. %, more preferably at least 25 wt. % of the composition.
106. The process as in any of the preceding embodiments, wherein in step (vi) the excipients of component (c) comprise, or optionally consist of, at least 60 wt. %, preferably at least 75 wt. %, more preferably at least 85 wt. %, of a water-soluble sugar or sugar alcohol.
107. The process as in embodiment 106, wherein the water-soluble sugar or sugar alcohol is selected from sucrose, sorbitol, xylitol, erythritol, maltitol, isomaltitol, mannitol, and mixtures thereof.
108. The process as in any of the preceding embodiments, wherein in step (vi) the composition comprises as further excipients of component (d) citric acid, monosodium citrate and/or disodium citrate, more particularly citric acid in combination with monosodium citrate.
109. The process as in any of the preceding embodiments, wherein in step (vi) the amounts of the extended-release particles of component (a) and of the non-extended-release particles of component (b) are selected such that over the course of 4 hours, preferably over the course of 5 hours, more preferably over the course of 6 hours, release not more than 80% of the magnesium comprised in the composition, measured in a release apparatus with paddle stirrer (USP Dissolution Apparatus 2) in 900 mL of 0.1 N hydrochloric acid at 37° C. and a stirring speed of 75 rpm.
110. The process as in any of the preceding embodiments, wherein at least 60 wt. %, or at least 75 wt. %, or at least 85 wt. %, of the total magnesium comprised in the composition is comprised in the extended-release particles of component (a).
111. The process as in any of the preceding embodiments, wherein the composition comprises at least 5 wt. %, or at least 10 wt. %, or at least 12 wt. % of the total magnesium comprised in the composition in non-extended-release form.
112. The process as in any of the preceding embodiments, wherein the components of (b), (c) and/or (d) that are admixed in step (vi) are present optionally in granulated form.
113. The process as in any of the preceding embodiments, wherein the excipients of components (c) and (d) that are admixed in step (vi) comprise not more than 5 wt. % of excipients sparingly soluble in water.
114. The process as in any of the preceding embodiments, wherein the process air temperature in steps (iii) and/or (iv) is between 20 and 60° C., or between 20 and 50° C., or between 20 and 45° C.; for example, around 25° C. or 35° C.
115. The process as in any of the preceding embodiments, wherein the molten coating material provided in step (ii) is sprayed in step (iv) at a melting temperature of between 70 and 120° C., or between 75 and 110° C., or between 80 and 100° C. for example, at around 90 to 100° C.
116. The process as in any of the preceding embodiments, wherein in step (iv) a spray air temperature of between 70 and 130° C., or between 75 and 125° C., or between 80 and 120° C. is used; for example, around 100° C. or 120° C.
117. The process as in any of the preceding embodiments, wherein the spraying pressure in step (iv) is between 0.5 and 1.5 bar, or between 0.6 and 1.3 bar, or between 0.7 and 1.2 bar; for example, it is 0.8 to 1.2 bar, 0.8 to 0.9 bar or 1.0 to 1.2 bar.
118. The process as in any of the preceding embodiments, wherein the spray amount of the molten coating material per kilogram of core material for coating in step (iv) is between 2.0 and 10.0 g/kg/min, or between 2.5 and 9.0 g/kg/min, or between 3.0 and 8.0 g/kg/min; for example, around 3.3 g/kg/min.
119. The process as in any of the preceding embodiments, wherein the process, more particularly the coating steps (iii) to (v), is/are carried out in a fluidized bed apparatus on the laboratory scale (for example, in a Ventilus® V-2.5), and wherein the spray amount of the molten coating material in step (iv) is between 5.0 and 8.0 g/min, or between 5.5 and 7.5 g/min, or between 6.0 and 7.0 g/min; for example, is around 6.5 g/min.
120. The process as in any of the preceding embodiments, wherein in a further step, subsequent to step (vi), single doses of the composition are packaged into stick packs, sachets, glass ampoules or plastic ampoules.
121. A solid pharmaceutical or nutraceutical composition in the form of direct oral granules with at least dual release of active ingredient, produced by the process as in embodiments 67 to 120.

### EXAMPLES

### Example 1: Comparison of the Properties of Different MgO Cores

Granulated magnesium oxide (MgO) particles from various suppliers were investigated for properties including their particle size and their abrasion resistance, to examine which product is suitable for use as a core of the extended-release particles in a hotmelt coating process; they included dry-compacted unfinished products such as the ‘Heavy Magnesium Oxide EP’ from Kyowa Chemical Industry Co., Ltd Japan (identified hereinafter as ‘MgO 1’) and MagGran® MO from Magnesia GmbH Deutschland (hereinafter ‘MgO 2’).

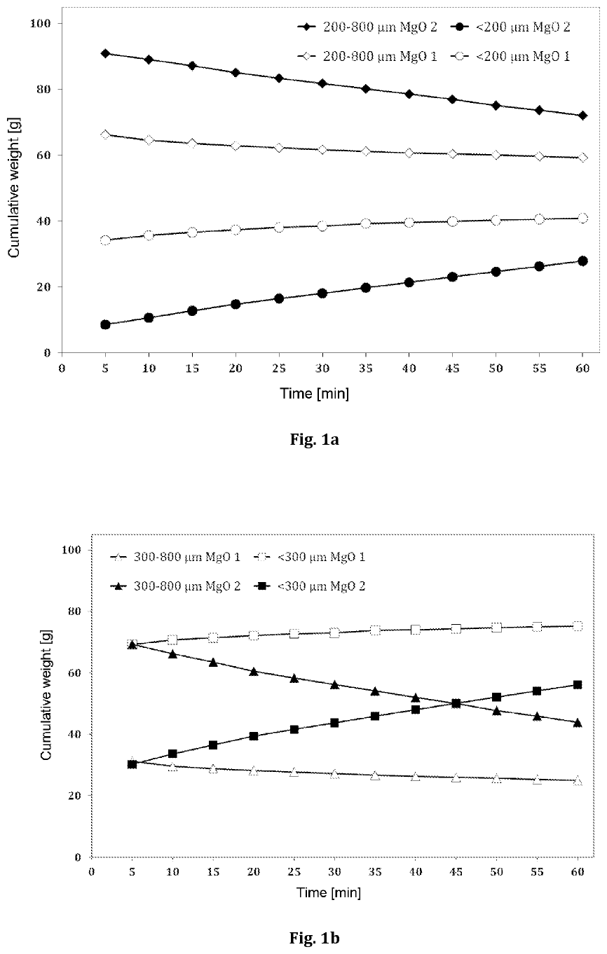

To simulate the abrasion of the MgO cores during hotmelt coating in the fluidized bed, they were subjected to mechanical stress for a total of 60 minutes on a screening tower (here, for example, a Retsch AS 200 basic, Retsch GmbH, Haan) with screen diameter of around 20 cm, using additionally a screening aid in the form of abrasion-resistant silica gel beads having a diameter of around 2-5 mm (here, for example, around 3 mm) and a bulk density of around 650-850 kg/m³ (here approximately 750 kg/m³). First of all, a sample amount of around 100 g of the MgO cores was applied to the topmost screen (800 μm), plus 1 g of screening aid per screen tray. The screening was carried out using a vibration amplitude of 80/min. Every 5 minutes, the individual screens (800 μm, 600 μm, 500 μm, 400 μm, 300 μm, 200 μm, 100 μm, and residue) and also the amount of MgO cores situated on them were weighed. The data for the two screen analyses are set out in tables 1a (MgO 1) and 1b (MgO 2) and also in FIGS. 1a and 1b.

TABLE 1a
Screen analysis of the MgO 1 cores

| Screen | Screening duration [min] | | | | | | | | | | | |
|---|---|---|---|---|---|---|---|---|---|---|---|---|
| [μm] | 5 | 10 | 15 | 20 | 25 | 30 | 35 | 40 | 45 | 50 | 55 | 60 |
| 800 | 0.0 | 0.0 | 0.0 | 0.0 | 0.0 | 0.0 | 0.0 | 0.0 | 0.0 | 0.0 | 0.0 | 0.0 |
| 600 | 0.0 | 0.0 | 0.0 | 0.0 | 0.0 | 0.0 | 0.0 | 0.0 | 0.0 | 0.0 | 0.0 | 0.0 |
| 500 | 0.1 | 0.0 | 0.0 | 0.0 | 0.0 | 0.0 | 0.0 | 0.0 | 0.0 | 0.0 | 0.0 | 0.1 |
| 400 | 6.4 | 5.4 | 4.8 | 4.4 | 4.1 | 3.8 | 3.5 | 3.2 | 3.0 | 2.8 | 2.6 | 2.4 |
| 300 | 24.7 | 24.2 | 24.0 | 23.8 | 23.6 | 23.4 | 23.2 | 23.1 | 23.0 | 22.9 | 22.8 | 22.6 |
| 200 | 35.0 | 35.0 | 34.8 | 34.7 | 34.6 | 34.5 | 34.5 | 34.4 | 34.4 | 34.4 | 34.4 | 34.3 |
| 100 | 33.3 | 34.6 | 35.4 | 36.1 | 36.6 | 36.9 | 37.4 | 37.6 | 37.8 | 38.1 | 38.3 | 38.5 |
| <100 | 0.9 | 1.1 | 1.2 | 1.3 | 1.5 | 1.6 | 1.9 | 2.0 | 2.1 | 2.2 | 2.3 | 2.4 |

TABLE 1b
Screen analysis of MgO 2 cores

| Screen | Screening duration [min] | | | | | | | | | | | |
|---|---|---|---|---|---|---|---|---|---|---|---|---|
| [μm] | 5 | 10 | 15 | 20 | 25 | 30 | 35 | 40 | 45 | 50 | 55 | 60 |
| 800 | 0.1 | 0.1 | 0.1 | 0.1 | 0.0 | 0.1 | 0.1 | 0.1 | 0.1 | 0.1 | 0.0 | 0.1 |
| 600 | 14.6 | 12.3 | 11.0 | 9.8 | 9.0 | 8.3 | 7.7 | 7.2 | 6.6 | 6.1 | 5.7 | 5.3 |
| 500 | 18.1 | 17.5 | 16.7 | 15.8 | 15.2 | 14.5 | 13.8 | 13.2 | 12.6 | 11.9 | 11.4 | 10.7 |
| 400 | 15.7 | 15.4 | 14.9 | 14.3 | 13.8 | 13.3 | 12.8 | 12.2 | 11.7 | 11.1 | 10.6 | 10.1 |
| 300 | 20.9 | 21.0 | 20.9 | 20.6 | 20.3 | 20.1 | 19.8 | 19.4 | 19.1 | 18.6 | 18.2 | 17.8 |
| 200 | 21.6 | 22.9 | 23.7 | 24.6 | 25.1 | 25.6 | 26.1 | 26.6 | 27.0 | 27.4 | 27.8 | 28.2 |
| 100 | 5.6 | 6.8 | 7.9 | 8.9 | 9.7 | 10.4 | 11.2 | 11.9 | 12.6 | 13.3 | 14.0 | 14.7 |
| <100 | 3.0 | 3.9 | 4.9 | 5.9 | 6.8 | 7.7 | 8.6 | 9.5 | 10.5 | 11.4 | 12.3 | 13.2 |

Alternatively to this, the abrasion resistance of the MgO cores was also determined by means of a suction intake experiment in which 400 g of the MgO cores were taken in by suction, using reduced pressure, into a fluidized bed apparatus on the laboratory scale, and were fluidized dry in this apparatus subsequently for around 5 min at a temperature of 25° C. with an air quantity of 35 m³/h. In this example, a Ventilus® V-2.5 apparatus was used for this experiment (Romaco Innojet, Steinen, Germany).

In addition to this, in both experiments (screen analysis and suction intake experiment), the particle size of the cores used was determined by means of dynamic image analysis, in a Camsizer® XT apparatus, equipped with the X-Jet plug-in cartridge and also with the associated analytical software (Retsch Technology GmbH, Haan, Germany). The corresponding data—determined in each case from the volume distribution—are listed in table 2:

TABLE 2
Particle sizes of two different unfinished MgO core products

| | MgO 1 | | | MgO 2 | | |
|---|---|---|---|---|---|---|
| | a | b | c | a | b | c |
| D10 | 88.0 | 99.9 | 75.7 | 53.2 | 28.0 | 18.3 |
| D50 | 223.4 | 225.4 | 222.3 | 354.7 | 264.5 | 294.4 |
| Mean | 234.4 | 234.7 | 231.4 | 358.2 | 285.0 | 307.3 |
| D90 | 399.4 | 390.2 | 397.6 | 644.3 | 588.3 | 630.4 |
| Scatter | 118.7 | 111.9 | 119.1 | 211.4 | 204.8 | 228.5 |
| (D90 − D10)/D50 | 1.39 | — | — | 1.67 | — | — |

a unstressed (unfinished MgO core product)
b abrasion after 60 min in screening tower with screening aid (2-5 mm silica gel; ρ = 750 kg/m³)
c abrasion after 5 min in Ventilus suction intake experiment and fluidization

As can be read off from the Camsizer data, the unfinished MgO 2 product of the magnesium oxide-containing cores is significantly more susceptible to abrasion and/or fracture of the cores than is the unfinished MgO 1 product and exhibits a reduction in the values for all four parameters (D10, D90, median D50 and mean of the volume-based particle size distribution). In contrast to this, the values for the unfinished MgO 1 product are predominantly stable within the frame of the measurement scatter, which suggests a fairly low propensity for dust abrasion or fracture of the cores.

The same tendency is also evident from FIGS. 1a and 1b: while the fine fraction (<200 μm in FIG. 1a, and <300 μm in FIG. 1b) for the MgO 2 cores rises constantly and approximately linearly and, consequently, the mass of cores in the respective particle size classes 200-800 μm and 300-800 μm, respectively, falls constantly over the course of the 60 min screening duration, for the MgO 1 cores, after an initial abrasion during the first 15-20 min, there is subsequently only very moderate abrasion recorded. Even in these first 15-20 min, however, the abrasion for the unfinished MgO 1 product is significantly lower than for the unfinished MgO 2 product.

As a consequence, the advantageous abrasion resistance of the MgO 1 cores, according to the invention, enables a melt coating process which was hitherto not possible with diverse other commercially available MgO cores, or was not possible reproducibly and not on an industrial scale (e.g., ≥50 kg per batch).

### Example 2: Production of Coated, Extended-Release MgO Particles on the Laboratory Scale

MgO 1 cores as in example 1 (here 0.8 kg) were coated in a fluidized bed apparatus (here a Ventilus V 2.5 equipped with a Romaco Innojet hotmelt apparatus IHD 1; Romaco Innojet, Steinen, Germany) in a hotmelt coating process with molten glycerol tristearate (here Dynasan® 118). The temperature of the melt was 90-100° C., the spraying pressure was around 0.8-0.9 bar, the spraying rate was around 6.5 g/min, and the spray air temperature was around 100° C.

In this way, coating levels of 30 wt. %, 20 wt. %, 15 wt. %, and 10 wt. % were produced, based on the weight of the coated, extended-release MgO particles (i.e., 42.9 wt. %, 25.0 wt. %, 17.6 wt. %, and 11.1 wt. %, based on the original weight of the uncoated MgO cores). An amount of around 921 mg of extended-release particles with a coating level of 10 wt. % therefore contains around 829 mg of MgO (corresponding to around 500 mg Mg) and around 92 mg of the Dynasan®118.

Table 3 sets out the particle sizes of the unfinished product (uncoated cores) by comparison with the coated batch with 10% coating level and indicates a coating approximately 20 μm thick. Moreover, the particle size distribution of the coated cores is somewhat narrower, as indicated by the somewhat lower quotient (D90-10)/D50.

TABLE 3
Particle sizes of MgO 1 cores (uncoated vs. coating level 10%)

| | MgO 1 | MgO 1 90:10 |
|---|---|---|
| D10 | 95.5 | 136.8 |
| D50 | 206.1 | 250.3 |
| Mean | 217.0 | 259.5 |
| D90 | 359.5 | 401.5 |
| Scatter | 101.2 | 105.8 |
| (D90 − D10)/D50 | 1.28 | 1.06 |

### Example 3: Production of Coated, Extended-Release MgO Particles on the Industrial Scale

MgO 1 cores as in example 1 (here 76.5 kg) were coated in a fluidized bed apparatus (here a Ventilus V 100 equipped with a Romaco Innojet hotmelt apparatus IHD 1; Romaco Innojet, Steinen, Germany) in a hotmelt coating process with molten glycerol tristearate (here Dynasan® 118). The temperature of the melt was 90-100° C., the spraying pressure was around 1.0-1.2 bar, the spraying rate was around 250 g/min, and the spray air temperature was around 120° C.

In this way, coating levels of 30 wt. %, 20 wt. %, 15 wt. %, and 10 wt. % were produced, based on the weight of the coated, extended-release MgO particles (i.e., 42.9 wt. %, 25.0 wt. %, 17.6 wt. %, and 11.1 wt. %, based on the original weight of the uncoated MgO cores).

### Example 4: Production of Direct Oral Granule Formulations with Extended-Release MgO Particles

The extended-release MgO particles obtained in example 2, with a coating level of 15 wt. % or 10 wt. %, were admixed with various excipient mixtures (table 4 and table 5). The excipient mixtures were first combined separately and then admixed to the extended-release MgO particles. The weight fraction of the extended-release MgO particles as a proportion of the total weight of the final mixture, the final direct oral granules, here is around 57 wt. % for the mixture of table 4, and around 34 wt. % for the mixture of table 5.

The direct oral granules ODG 1-3 contain respectively around 500 mg of magnesium oxide in extended-release particles, 7.85 mg of trimagnesium dicitrate in the form of non-extended-release particles, and also 10 μg of Vit. D3 and 20 μg of Vit. B12. The three oral granules differ primarily in their taste imparters such as the flavors, sugar alcohols and/or sweeteners.

The direct oral granules ODG 4 contain around 350 mg of magnesium oxide in extended-release particles, 42.15 mg of magnesium hydrogen citrate and 7.85 mg of trimagnesium dicitrate (both in the form of non-extended-release particles), and also 1.1 mg of Vit. B1, 1.4 mg of Vit. B2, 1.4 mg of Vit. B6, and 2.5 μg of Vit. B12.

TABLE 4
Composition of the components admixed
to component (a) (figures in %)

| | ODG 1 | ODG 2 | ODG 3 |
|---|---|---|---|
| Constituents | MgO 1 85:15 | MgO 1 85:15 | MgO 1 90:10 |
| Sorbitol | 35.98 | 35.98 | 37.26 |
| Xylitol | 27.60 | 27.60 | 19.15 |
| Monosodium citrate | 7.59 | 7.59 | 14.73 |
| (anhydrous) | | | |
| Calcium-effervescent | 6.90 | 6.90 | 7.37 |
| base | | | |
| Trimagnesium | 6.90 | 6.90 | 7.37 |
| dicitrate (anhydrous) | | | |
| Citric acid | 4.83 | 4.83 | 2.95 |
| Cyanocobalamin | 3.17 | 3.17 | 3.39 |
| (Vit. B12) 0.1% | | | |
| Carmellose sodium | 2.76 | 2.76 | 2.95 |
| (Na-CMC) | | | |
| Lemon flavor | 2.76 | — | 2.95 |
| Multifruit flavor | — | 2.76 | — |
| Cholecalciferol | 0.75 | 0.75 | 0.80 |
| (Vit. D3) | | | |
| Magnesium stearate | 0.55 | 0.55 | 0.59 |
| Aspartame | 0.21 | 0.21 | 0.44 |
| Acesulfame-K | — | — | 0.07 |

TABLE 5
Composition of the components admixed
to component (a) (figures in %)

| | ODG 4 |
|---|---|
| Constituents | MgO 1 90:10 |
| Magnesium hydrogen citrate | 39.51 |
| Sorbitol | 43.81 |
| Xylitol | 7.97 |
| Trimagnesium dicitrate | 3.98 |
| (anhydrous) | |
| Carmellose sodium | 1.59 |
| Orange flavor | 1.60 |
| Pyridoxin-HCl (Vit. B1) | 0.47 |
| Riboflavin (Vit. B2) | 0.38 |
| Thiamin nitrate (Vit. B6) | 0.37 |
| Cyanocobalamin (Vit. B12) 0.1% | 0.23 |
| Sucralose | 0.09 |

### Example 5: Release Behavior in 0.1 N Hydrochloric Acid

The release behavior of magnesium from the melt-coated extended-release particles of example 2 with a coating level of 10 wt. % (90:10) and 15 wt. % (85:15) and also from the direct oral granules of the invention formulated with said particles, from example 4, was determined in a pharmacopeia paddle stirrer release apparatus (USP Dissolution Apparatus 2) at 37° C. in 900 mL of 0.1 N hydrochloric acid (HCl). The stirring speed was 75 rpm. All of the experiments were repeated at least six times, and mean values and standard deviations were calculated.

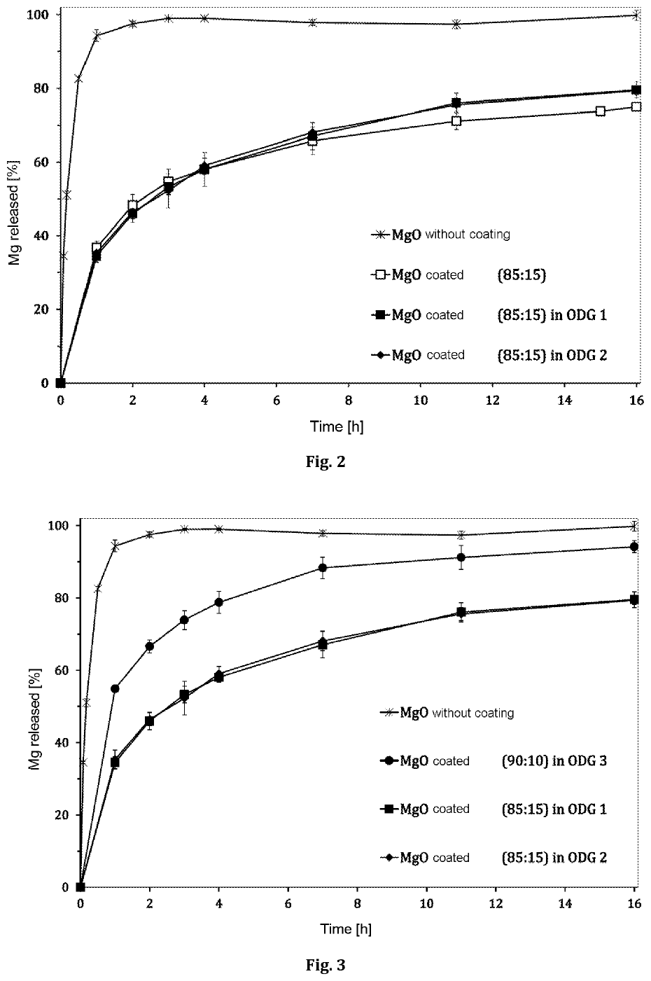

As is evident from FIG. 2, the release from melt-coated MgO 1 cores at a coating level of 15 wt. % is sufficiently extended to ensure a stable supply of magnesium over the entire day. Moreover, the release profiles of the two compositions of the invention, ODG 1 and ODG 2, are very similar to the release profile of the mere melt-coated MgO 1 cores, indicating that the rest of the constituents of the oral granules do not adversely affect the release from the extended-release particles.

As is evident from FIG. 3, the release from melt-coated MgO 1 cores at a coating level of only 10 wt. % is, as expected, somewhat faster than at a coating level of 15 wt. %; the release can therefore be adjusted with the coating level. Both, however, are sufficiently release-extended to ensure a stable supply of magnesium over the entire day.

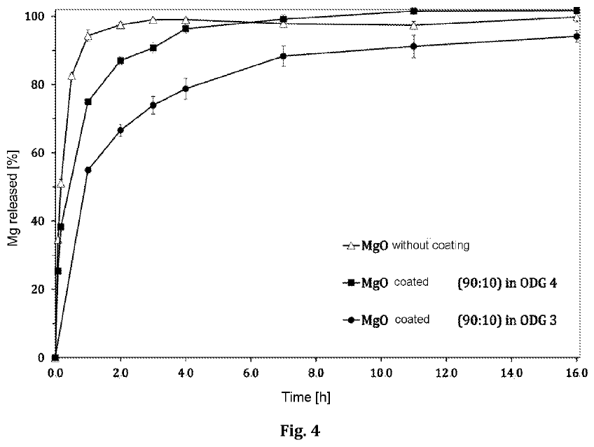

FIG. 4 shows the release from melt-coated MgO 1 cores at a coating level of 10 wt. % for direct oral granules containing around 50 mg of non-extended-release magnesium and around 350 mg of extended-release magnesium in the form of the melt-coated MgO 1 cores (ODG 4), and also, in comparison thereto, direct oral granules which contain only around 8 mg of non-extended-release magnesium and around 500 mg of extended-release magnesium in the form of the melt-coated MgO 1 cores (ODG 3). As is evident from FIG. 4, the release of magnesium from ODG 4, owing to the higher non-extended-release fraction it contains, is quicker than for ODG 3, with predominantly release-extended magnesium fractions. This shows that the release of magnesium can be adapted not only via the coating level but also through the choice of the proportions of extended-release and non-extended-release magnesium to one another.

1. A solid pharmaceutical or nutraceutical composition in the form of direct oral granules with at least dual release of active ingredient, comprising components as follows:
coated, extended-release particles having a magnesium-containing core and a lipid coating, the core comprising or consisting of a magnesium compound, and the extended-release particles releasing the comprised magnesium in an extended manner;
non-extended-release, magnesium-containing particles, the particles comprising or consisting of a magnesium compound, and the particles releasing the comprised magnesium in a non-extended manner;
one or more water-soluble excipients selected from the group of sugars, sugar alcohols, and oligosaccharides; and
optionally one or more further excipients;
wherein at least the magnesium-containing cores of the extended-release particles of component (a), and/or a sample thereof, have an abrasion resistance such that after vibration screening over a period of 60 minutes:
the fine fraction of particles having a screen diameter of <200 μm after a screening time of 60 minutes, t₆₀ₘᵢₙ, is higher by not more than 100 wt. %, preferably by not more than 50 wt. %, more preferably by not more than 20 wt. % relative to the initial fine fraction of this screen diameter after a screening time of 5 minutes, t₅ₘᵢₙ; and/or
the fraction of particles having a screen diameter of 200 μm≤x<800 μm after a screening time of 60 minutes, t₆₀ₘᵢₙ, is lower by not more than 18 wt. %, preferably by not more than 15 wt. %, more preferably by not more than 12 wt. %, relative to the initial fraction of this screen diameter after a screening time of 5 minutes, t₅ₘᵢₙ.

2. The composition according to claim 1, wherein the vibration screening is carried out at a vibration amplitude of around 50/min to around 100/min, for example, around 80/min, and/or using a screening tower having screen trays of around 10-22 cm screen tray diameter, and/or using a screening tower having screen trays of around 10-22 cm screen tray diameter, and also an initial test amount of around 50-150 g of the magnesium-containing cores, for example, around 100 g, and further optionally wherein in the screening tower the screens having a screen diameter of 800 μm, 600 μm, 500 μm, 400 μm, 300 μm, 200 μm, and 100 μm and also the bottom cup are used.

3. (canceled)

4. (canceled)

5. The composition according to claim 1, wherein the vibration screening is carried out using a screening aid in the form of abrasion-resistant beads having a diameter of around 2-6 mm, for example, around 3 mm; preferably a screening aid in the form of abrasion-resistant silica gel beads having a diameter of around 2-6 mm, for example, around 3 mm, optionally a screening aid in the form of abrasion-resistant silica gel beads having a bulk density of around 650-850 kg/m³, for example, having a bulk density of around 750 kg/m³.

6. (canceled)

7. The composition according to claim 1, wherein the vibration screening is carried out using per screen tray around 0.5-2.0 g of the screening aid, for example, around 1 g.

8. (canceled)

9. The composition according to claim 1, wherein the vibration screening is carried out with 100 g of the magnesium-containing cores and with 1 g of screening aid per screen tray and also at a vibration amplitude of 80/min.

10. The composition according to claim 1, wherein the magnesium-containing cores of the extended-release particles have a unimodal particle size distribution, and optionally a narrow particle size distribution in which the quotient (D90-D10)/D50 is less than 1.60, preferably less than 1.50, and more preferably less than 1.40.

11. The composition according to claim 1, wherein at least the magnesium compound in the extended-release particles of component (a) in its anhydrous form has a magnesium content of 15 wt. % or more, preferably 25 wt. % or more, more preferably 50 wt. % or more.

12. (canceled)

13. The composition according to claim 1, wherein the coating of the extended-release particles comprises, or substantially consists of, a lipid having a melting point of at least 50° C., preferably a lipid having a melting point of at least 60° C.

14. (canceled)

15. (canceled)

16. (canceled)

17. (canceled)

18. (canceled)

19. (canceled)

20. The composition according to claim 1, wherein the extended-release particles of component (a) make up between 15 and 85 wt. %, or between 25 and 75 wt. %, or between 30 and 70 wt. %, or between 30 and 60 wt. % of the composition.

21. A method for characterizing a magnesium-containing core for producing a solid, lipid-coated pharmaceutical or nutraceutical composition in a fluidized bed, the core comprising or consisting of a magnesium compound, and the method being carried out by means of vibration screening, and by magnesium-containing cores which are characterized as being suitable for producing the solid, lipid-coated pharmaceutical or nutraceutical composition in a fluidized bed in this test having an abrasion resistance such that after the vibration screening over a period of 60 minutes:
the fine fraction of particles having a screen diameter of <200 μm after a screening time of 60 minutes, t₆₀ₘᵢₙ, is higher by not more than 100 wt. %, preferably by not more than 50 wt. %, more preferably by not more than 20 wt. %, relative to the initial fine fraction of this screen diameter after a screening time of 5 minutes, t₅ₘᵢₙ; and/or
the fraction of particles having a screen diameter of 200 μm≤x<800 μm after a screening time of 60 minutes, t₆₀ₘᵢₙ, is lower by not more than 18 wt. %, preferably is lower by not more than 15 wt. %, more preferably is lower by not more than 12 wt. %, relative to the initial fraction of this screen diameter after a screening time of 5 minutes, t₅ₘᵢₙ.

22. The method according to claim 21, wherein the vibration screening is carried out at a vibration amplitude of around 50/min to around 100/min, for example, at a vibration amplitude of around 80/min, and/or wherein the vibration screening is carried out using a screening tower having screen trays of around 10-22 cm screen tray diameter, for example 20 cm, and optionally also an initial test amount of around 50-150 g of the magnesium-containing cores, for example, around 100 g.

23. (canceled)

24. (canceled)

25. (canceled)

26. The method according to claim 21, wherein the vibration screening is carried out using a screening aid in the form of abrasion-resistant beads having a diameter of around 2-6 mm; for example, around 3 mm, and/or having a bulk density of around 650-850 kg/m³, optionally wherein the abrasion-resistant beads are silica gel beads.

27. (canceled)

28. (canceled)

29. The method according to claim 21, wherein a screening aid in the form of abrasion-resistant silica gel beads having a diameter of around 2-6 mm is used; for example, around 3 mm, and/or having a bulk density of around 650-850 kg/m³ is used, for example, a bulk density of around 750 kg/m³.

30. (canceled)

31. (canceled)

32. The method according to claim 21, wherein per screen tray around 0.5-2.0 g of the screening aid is used, for example, around 1 g, and optionally wherein the screening aid is in the form of abrasion-resistant silica gel beads having a diameter of around 2-6 mm (e.g. 3 mm) and a bulk density of around 650-850 kg/m³ is used, e.g., a bulk density of around 750 kg/m³.

33. (canceled)

34. The method according to claim 21, wherein the vibration screening is carried out using a screening tower having screen trays of around 10-22 cm screen tray diameter, and wherein in the screening tower the screens having a screen diameter of 800 μm, 600 μm, 500 μm, 400 μm, 300 μm, 200 μm, and 100 μm and also the bottom cup are used.

35. The method according to claim 21, wherein the following are used for the vibration screening:
a screening tower having screens having a screen tray diameter of around 10-22 cm and screen diameters of 800 μm, 600 μm, 500 μm, 400 μm, 300 μm, 200 μm, and 100 μm, and also the bottom cup,
an initial test quantity of the magnesium-containing cores of around 50-150 g,
around 0.5-2.0 g of screening aid per screen tray,
the abrasion-resistant silica gel beads which are used as screening aid having a diameter of around 2-6 mm and a bulk density of around 650-850 kg/m³.

36. The method according to claim 21, wherein the following are used for the vibration screening:
a screening tower having screens having a screen tray diameter of around 10-22 cm and screen diameters of 800 μm, 600 μm, 500 μm, 400 μm, 300 μm, 200 μm, and 100 μm, and also the bottom cup,
an initial test quantity of the magnesium-containing cores of around 75-125 g,
around 0.7-1.5 g of screening aid per screen tray,
the abrasion-resistant silica gel beads which are used as screening aid having a diameter of around 2-5 mm and a bulk density of around 700-800 kg/m³.

37. The method according to claim 21, wherein the following are used for the vibration screening:
a screening tower having screens having a screen tray diameter of around 20 cm and screen diameters of 800 μm, 600 μm, 500 μm, 400 μm, 300 μm, 200 μm, and 100 μm, and also the bottom cup,
an initial test quantity of the magnesium-containing cores of around 100 g,
around 1 g of screening aid per screen tray,
the abrasion-resistant silica gel beads which are used as screening aid having a diameter of around 2-4 mm and a bulk density of around 750 kg/m³.

38. The method according to claim 21, wherein the magnesium compound in the magnesium-containing cores is selected from magnesium oxide (MgO), magnesium carbonate (MgCO₃), magnesium chloride (MgCl₂), magnesium hydrogen phosphate (MgHPO₄) and/or magnesium acetate (Mg(CH₃COO)₂), preferably wherein the magnesium compound in the magnesium-containing cores is magnesium oxide (MgO).

39. (canceled)

40. The method according to claim 1, wherein magnesium-containing cores which in this test have an abrasion resistance such that after the vibration screening over a period of 60 minutes:
the fine fraction of particles having a screen diameter of <200 μm after a screening time of 60 minutes, t₆₀ₘᵢₙ, is higher by not more than 100 wt. %, preferably by not more than 50 wt. %, more preferably by not more than 20 wt. %, relative to the initial fine fraction of this screen diameter after a screening time of 5 minutes, t₅ₘᵢₙ; and/or
the fraction of particles having a screen diameter of 200 μm≤x<800 μm after a screening time of 60 minutes, t₆₀ₘᵢₙ, is lower by not more than 18 wt. %, preferably by not more than 15 wt. %, more preferably by not more than 12 wt. %, relative to the initial fraction of this screen diameter after a screening time of 5 minutes, t₅ₘᵢₙ
are suitable especially for producing the solid, lipid-coated pharmaceutical or nutraceutical composition by means of a hotmelt coating process in a fluidized bed.

41. A process for producing a solid pharmaceutical or nutraceutical composition according to claim 1, comprising the following steps:
(i) providing a magnesium-containing core, the core comprising or consisting of a magnesium compound;
(ii) providing a molten coating material comprising a lipid;
(iii) fluidizing the magnesium-containing cores;
(iv) spraying the fluidized magnesium-containing cores with the molten coating material;
(v) cooling the coated magnesium-containing cores, so that the lipid solidifies and extended-release particles of component (a) are obtained which release the comprised magnesium in an extended manner; and
(vi) mixing the extended-release particles obtained in this way as component (a) with the following further components:
(b) non-extended-release particles having a second magnesium-containing core, the particle comprising or consisting of a magnesium compound, and the particles releasing the comprised magnesium in a non-extended manner;
(c) one or more water-soluble excipients selected from the group of sugars, sugar alcohols, and oligosaccharides; and
(d) optionally further excipients;
wherein at least the magnesium-containing cores of the extended-release particles of component (a), and/or a sample thereof, according to the characterization method according to claim 21, are suitable for producing the solid, lipid-coated pharmaceutical or nutraceutical composition, thus have an abrasion resistance such that after vibration screening over a period of 60 minutes:
the fine fraction of particles having a screen diameter of <200 μm after a screening time of 60 minutes, t₆₀ₘᵢₙ, is higher by not more than 100 wt. %, preferably by not more than 50 wt. %, more preferably by not more than 20 wt. % relative to the initial fine fraction of this screen diameter after a screening time of 5 minutes, t₅ₘᵢₙ; and/or
the fraction of particles having a screen diameter of 200 μm≤x<800 μm after a screening time of 60 minutes, t₆₀ₘᵢₙ, is lower by not more than 18 wt. %, preferably by not more than 15 wt. %, more preferably by not more than 12 wt. % relative to the initial fraction of this screen diameter after a screening time of 5 minutes, t₅ₘᵢₙ.

42. A solid pharmaceutical or nutraceutical composition in the form of direct oral granules with at least dual release of active ingredient, produced by the process according to claim 41.
